(19) United States
(12) Patent Application Publication
(10) Pub. No.: US 2024/0383958 A1
(43) Pub. Date: Nov. 21, 2024

(54) APELIN RECEPTOR AGONISTS AND USES THEREOF

(71) Applicant: VISTERRA, INC., Waltham, MA (US)

(72) Inventors: Karthik Viswanathan, Waltham, MA (US); Brahm Yachnin, Waltham, MA (US); Laura Vinue-Santolalla, Waltham, MA (US)

(73) Assignee: VISTERRA, INC., Waltham, MA (US)

(21) Appl. No.: 18/633,222

(22) Filed: Apr. 11, 2024

Related U.S. Application Data
(60) Provisional application No. 63/585,027, filed on Sep. 25, 2023.
(60) Provisional application No. 63/495,388, filed on Apr. 11, 2023.

(51) Int. Cl. C07K 14/575; C07K 14/765; C07K 16/18

(52) U.S. Cl. CPC C07K 14/575; C07K 14/765; C07K 16/18; C07K 2319/30

(57) ABSTRACT

The present invention provides, among other things, apelin receptor agonists with increased half-life, reduced desensitization, improved apelin receptor signaling properties (e.g., bias for Gα signaling over β-arrestin signaling), enhanced stability, and/or enhanced binding to apelin receptor.

### CROSS-REFERENCE TO RELATED APPLICATIONS

The present application claims priority to U.S. Provisional Application Ser. No. 63/585,027, filed Sep. 25, 2023, and to U.S. Provisional Application Ser. No. 63/495,388, filed Apr. 11, 2023, the disclosures of which are hereby incorporated by reference in their entireties.

### INCORPORATION-BY-REFERENCE OF SEQUENCE LISTING

The instant application contains a sequence listing which has been submitted electronically in XML format and is hereby incorporated by reference in its entirety. The sequence listing file, entitled SVI-005US1_SL.xml, was created on Apr. 11, 2024, and is 76,319 bytes in size.

### BACKGROUND

The apelin system is an endogenous physiological regulator that is emerging as a potential therapeutic target for many diseases. This system comprises the apelin receptor and its two families of endogenous ligands, apelin and elabela/toddler (ELA; also known as apelin receptor early endogenous ligand). The APLNR gene (also known as APJ) was identified and cloned in 1993. This gene was found to encode the apelin receptor, a novel G protein-coupled receptor (GPCR) that has ˜50% homology with the type 1 angiotensin II (AT1) receptor. The apelin receptor system opposes the actions of angiotensin II (Ang II) agonism at the AT1 receptor in vitro and in vivo. The apelin receptor is highly conserved between species with ˜90% sequence similarity between mouse, rat, and human proteins.

As apelin peptides have a half-life of only a few minutes in humans, and rapid receptor desensitization via coupling to β-arrestins has been reported, clinical studies of the apelin system are challenging. These issues have led to efforts to produce apelin receptor agonists that may be used as pharmacological probes to explore the role of the system in health and disease.

### SUMMARY OF THE INVENTION

The present invention provides, among other things, improved apelin receptor agonists and uses of such agonists in effectively treating diseases such as pulmonary arterial hypertension (PAH), systemic sclerosis (SSc), heart failure with preserved ejection fraction (HfpEf), age-associated sarcopenia, and acute kidney injury (AKI).

As described herein, the present invention is, in part, based on the identification of a new class of apelin or elabela analogues that have increased half-life, reduced desensitization, improved apelin receptor signaling properties (e.g., bias for Gα signaling over β-arrestin signaling), enhanced stability, and/or enhanced binding to apelin receptor. This is significant because the apelin receptor agonists of the present invention can effectively elicit response to desired downstream apelin receptor pathways without significantly adversely affecting the internalization and transcriptional downregulation apelin receptors after treatment with apelin receptor agonists. Inventive apelin receptor agonists of the present invention promise a more potent treatment of apelin receptor-mediated diseases and disorders including pulmonary arterial hypertension (PAH), systemic sclerosis (SSc), heart failure with preserved ejection fraction (HfpEf), age-associated sarcopenia, and acute kidney injury (AKI).

In one aspect, the present invention provides, among other things, an apelin receptor agonist comprising an apelin analog, wherein the apelin analog comprises one or more amino acid substitutions as compared to SEQ ID NO: 1 (QRPRLSHKGPMPF), wherein the one or more amino acid substitution occurs at position 1, 6, 8, 9, 11, 12, and/or 13.

In some embodiments, the apelin receptor agonist comprises one or more amino acid substitutions selected from P12V, K8W, Q1E, M11H, M11A, F13Y, and/or P12A, P12H, F13W, S6Q, and/or G9N.

In one aspect, the present invention provides, among other things, an apelin receptor agonist comprising an apelin analogue, wherein the apelin analogue comprises the amino sequence: X1-R-P-R-L-S-H-X2-G-P-X3-X4-X5 (SEQ ID NO: 63), wherein, X1 is Q or E; X2 is K or W; X3 is M, H, or A; X4 is P, V, A, or H; and/or X5 is F, Y or W.

In one aspect, the present invention provides, among other things, an apelin receptor agonist comprising an apelin analogue, wherein the apelin analogue comprises the amino sequence: Q-R-P-R-L-Q-H-K-N-P-M-A-X1 (SEQ ID NO: 64), wherein, X1 is F or W.

In one aspect, the present invention provides, among other things, an apelin receptor agonist comprising an apelin analogue, wherein the apelin analogue comprises the amino sequence: Q-R-P-R-L-S-H-K-G-P-X1-X2-X3 (SEQ ID NO: 65), wherein, X1 is M, H, or A; X2 is P, V, A, or H; and/or X3 is F, Y, or W.

In one aspect, the present invention provides, among other things, an apelin receptor agonist comprising an apelin analogue, wherein the apelin analogue comprises the amino sequence: X1-R-P-R-L-X2-H-X3-X4-P-X5-X6-X7 (SEQ ID NO: 66), wherein, X1 is Q or E; X2 is S or Q; X3 is K or W; X4 is G or N; X5 is M, H, or A; X6 is P, V, A or H; and/or X7 is F, Y or W.

In some embodiments, the apelin analog comprises the amino acid sequence of SEQ ID NO: 1 (QRPRLSHKGPMPF). In some embodiments, the apelin analog comprises the amino acid sequence of SEQ ID NO: 2 (QRPRLSHKGPMVF). In some embodiments, the apelin analog comprises the amino acid sequence of SEQ ID NO: 3 (QRPRLSHWGPMPF). In some embodiments, the apelin analog comprises the amino acid sequence of SEQ ID NO: 4 (ERPRLSHKGPMPF). In some embodiments, the apelin analog comprises the amino acid sequence of SEQ ID NO: 5 (QRPRLSHKGPHPF). In some embodiments, the apelin analog comprises the amino acid sequence of SEQ ID NO: 6 (QRPRLSHKGPAPY). In some embodiments, the apelin analog comprises the amino acid sequence of SEQ ID NO: 7 (QRPRLSHKGPMAF). In some embodiments, the apelin analog comprises the amino acid sequence of SEQ ID NO: 50 (QRPRLSHKGPMHW). In some embodiments, the apelin analog comprises the amino acid sequence of SEQ ID NO: 51 (QRPRLSHKGPAAW). In some embodiments, the apelin analog comprises the amino acid sequence of SEQ ID NO: 52 (QRPRLSHKGPAAY). In some embodiments, the apelin analog comprises the amino acid sequence of SEQ ID NO: 55 (QRPRLSHWGPMAW). In some embodiments, the apelin analog comprises the amino acid sequence of SEQ ID NO: 53 (QRPRLQHKNPMAF). In some embodiments, the apelin analog comprises the amino acid sequence of SEQ ID NO: 54 (QRPRLQHKNPMAW).

In one aspect, the present invention provides, among other things, an apelin receptor agonist comprising an elabela analogue, wherein the elabela analogue comprises one or more amino acid substitutions as compared to SEQ ID NO: 8 (KLRKHNCLQRRCMPLHSRVPFP), wherein the one or more amino acid substitution occurs at position 3, 7, 8, 9, 10, 12, 14, 15, 17 and/or 18.

In some embodiments, the apelin analog comprises one or more amino acid substitutions comprise R3N, P14I, S17Q, S17R, Q9K, R18Y, L15Y, C7S, C12Y, R10E, Q9I, L8A, and/or Q9T.

In one aspect, the present invention provides, among other things, an apelin receptor agonist comprising an elabela analog, wherein the elabela analog comprises the amino sequence: K-L-Z1-K-H-N-Z2-Z3-Z4-Z5-R-Z6-M-Z7-Z8-H-Z9-Z10-V-P-F-P (SEQ ID NO: 67), wherein Z1 is R or N; Z2 is C or S; Z3 is A or L; Z4 is Q, I, K, or T; Z5 is R or E; Z6 is C or Y; Z7 is P or I; Z8 is L or Y; Z9 is S, Q, or R; and/or Z10 is R or Y.

In some embodiments, the elabela analog comprises the amino acid sequence of SEQ ID NO: 8 (KLRKHNCLQRRCMPLHSRVPFP). In some embodiments, the elabela analog comprises the amino acid sequence of SEQ ID NO: 9 (KLNKHNCLQRRCMPLHSRVPFP). In some embodiments, the elabela analog comprises the amino acid sequence of SEQ ID NO: 10 (KLRKHNCLQRRCMILHSRVPFP). In some embodiments, the elabela analog comprises the amino acid sequence of SEQ ID NO: 11 (KLRKHNCLQRRCMPLHQRVPFP). In some embodiments, the elabela analog comprises the amino acid sequence of SEQ ID NO: 12 (KLRKHNCLQRRCMPLHRRVPFP). In some embodiments, the elabela analog comprises the amino acid sequence of SEQ ID NO: 13 (KLRKHNCLKRRCMPLHSRVPFP). In some embodiments, the elabela analog comprises the amino acid sequence of SEQ ID NO: 30 (KLRKHNCLQRRCMPLHSYVPFP). In some embodiments, the elabela analog comprises the amino acid sequence of SEQ ID NO: 31 (KLRKHNCLQRRCMPYHSRVPFP). In some embodiments, the elabela analog comprises the amino acid sequence of SEQ ID NO: 32 (KLRKHNSLQRRYMPLHSRVPFP). In some embodiments, the elabela analog comprises the amino acid sequence of SEQ ID NO: 33 (KLRKHNCLQERCMPLHSRVPFP). In some embodiments, the elabela analog comprises the amino acid sequence of SEQ ID NO: 34 (KLRKHNCLIRRCMPLHSRVPFP). In some embodiments, the elabela analog comprises the amino acid sequence of SEQ ID NO: 35 (KLRKHNCATRRCMPLHSRVPFP).

In some embodiments, the apelin receptor agonist comprises an apelin analog or the elabela analog fused to a carrier moiety. In some embodiments, the N-terminus of the apelin analogue or the elabela analogue is fused to the carrier moiety. In some embodiments, the carrier moiety extends a half-life of the apelin receptor agonist. In some embodiments, the carrier moiety comprises an Fc domain, human serum albumin (HSA), an anti-HSA antibody or a fragment thereof, lipids, or PEGs. In some embodiments, the Fc domain is a monovalent Fc. In some embodiments, the Fc domain is a bivalent Fc.

In some embodiments, the anti-HSA antibody or a fragment there of is an anti-HSA Fab, a nanobody (VHH), an scFv, or a V-NAR. In some embodiments, the anti-HSA VHH comprises ALB-23.

In one aspect, the present invention provides, among other things, an apelin analog or an elabela analogue, wherein the N-terminus of the apelin analog or the elabela analogue is fused to a carrier moiety via a linker.

In some embodiments, the N-terminus of the apelin analog or the elabela analog is fused to the C-terminus of the carrier moiety. In some embodiments, the carrier moiety extends a half-life of the apelin receptor agonist. In some embodiments, the carrier moiety comprises an Fc domain, a human serum albumin (HSA), an anti-HSA antibody or a fragment thereof, lipids, or PEGs. In some embodiments, the Fc domain is a monovalent Fc. In some embodiments, the Fc domain is a bivalent Fc. In some embodiments, the Fc domain is a IgG1, IgG2, IgG3, or IgG4 isotype. In some embodiments, the Fc domain comprises one or more mutations as compared to the wild-type Fc domain. In some embodiments, the Fc domain comprises SEQ ID NO: 14 or SEQ ID NO:15. In some embodiments, the anti-HSA antibody or a fragment there of is an anti-HSA Fab, a nanobody (VHH), an scFv, or a V-NAR. In some embodiments, the anti-HAS VHH comprises ALB-23. In some embodiments, the linker comprises GGGGSGGGGSGGGGS (SEQ ID NO: 16).

In some embodiments, the apelin receptor agonist comprises an amino acid sequence that is at least 80%, 85%, 90%, 93%, 95%, 98%, 99% identical to SEQ ID NO: 17. In some embodiments, the apelin receptor agonist comprises an amino acid sequence that is at least 80%, 85%, 90%, 93%, 95%, 98%, 99% identical to SEQ ID NO: 18. In some embodiments, the apelin receptor agonist comprises an amino acid sequence that is at least 80%, 85%, 90%, 93%, 95%, 98%, 99% identical to SEQ ID NO: 19. In some embodiments, the apelin receptor agonist comprises an amino acid sequence that is at least 80%, 85%, 90%, 93%, 95%, 98%, 99% identical to SEQ ID NO: 20. In some embodiments, the apelin receptor agonist comprises an amino acid sequence that is at least 80%, 85%, 90%, 93%, 95%, 98%, 99% identical to SEQ ID NO: 21. In some embodiments, the apelin receptor agonist comprises an amino acid sequence that is at least 80%, 85%, 90%, 93%, 95%, 98%, 99% identical to SEQ ID NO: 22. In some embodiments, the apelin receptor agonist comprises an amino acid sequence that is at least 80%, 85%, 90%, 93%, 95%, 98%, 99% identical to SEQ ID NO: 23. In some embodiments, the apelin receptor agonist comprises an amino acid sequence that is at least 80%, 85%, 90%, 93%, 95%, 98%, 99% identical to SEQ ID NO: 24. In some embodiments, the apelin receptor agonist comprises an amino acid sequence that is at least 80%, 85%, 90%, 93%, 95%, 98%, 99% identical to SEQ ID NO: 25. In some embodiments, the apelin receptor agonist comprises an amino acid sequence that is at least 80%, 85%, 90%, 93%, 95%, 98%, 99% identical to SEQ ID NO: 26. In some embodiments, the apelin receptor agonist comprises an amino acid sequence that is at least 80%, 85%, 90%, 93%, 95%, 98%, 99% identical to SEQ ID NO: 27. In some embodiments, the apelin receptor agonist comprises an amino acid sequence that is at least 80%, 85%, 90%, 93%, 95%, 98%, 99% identical to SEQ ID NO: 28. In some embodiments, the apelin receptor agonist comprises an amino acid sequence that is at least 80%, 85%, 90%, 93%, 95%, 98%, 99% identical to SEQ ID NO: 29. In some embodiments, the apelin receptor agonist comprises an amino acid sequence that is at least 80%, 85%, 90%, 93%, 95%, 98%, 99% identical to SEQ ID NO: 36. In some embodiments, the apelin receptor agonist comprises an amino acid sequence that is at least 80%, 85%, 90%, 93%, 95%, 98%, 99% identical to SEQ ID NO: 37. In some embodiments, the apelin receptor agonist comprises an amino acid sequence that is at least 80%, 85%, 90%, 93%, 95%, 98%, 99% identical to SEQ ID NO: 38 In some embodiments, the apelin receptor agonist comprises an amino acid sequence that is at least 80%, 85%, 90%, 93%, 95%, 98%, 99% identical to SEQ ID NO: 39. In some embodiments, the apelin receptor agonist comprises an amino acid sequence that is at least 80%, 85%, 90%, 93%, 95%, 98%, 99% identical to SEQ ID NO: 40. In some embodiments, the apelin receptor agonist comprises an amino acid sequence that is at least 80%, 85%, 90%, 93%, 95%, 98%, 99% identical to SEQ ID NO: 41. In some embodiments, the apelin receptor agonist comprises an amino acid sequence that is at least 80%, 85%, 90%, 93%, 95%, 98%, 99% identical to SEQ ID NO: 56. In some embodiments, the apelin receptor agonist comprises an amino acid sequence that is at least 80%, 85%, 90%, 93%, 95%, 98%, 99% identical to SEQ ID NO: 57. In some embodiments, the apelin receptor agonist comprises an amino acid sequence that is at least 80%, 85%, 90%, 93%, 95%, 98%, 99% identical to SEQ ID NO: 58. In some embodiments, the apelin receptor agonist comprises an amino acid sequence that is at least 80%, 85%, 90%, 93%, 95%, 98%, 99% identical to SEQ ID NO: 59. In some embodiments, the apelin receptor agonist comprises an amino acid sequence that is at least 80%, 85%, 90%, 93%, 95%, 98%, 99% identical to SEQ ID NO: 60. In some embodiments, the apelin receptor agonist comprises an amino acid sequence that is at least 80%, 85%, 90%, 93%, 95%, 98%, 99% identical to SEQ ID NO: 61.

In some embodiments, the apelin receptor agonist has a G_{αi} signaling assay EC50 value of less than 60 nM, less than 30 nM, less than 20 nM, or less than 10 nM, as measured by the depletion of cAMP. In some embodiments, the apelin receptor agonist has a beta-Arrestin signaling assay EC50 value of less than 60 nM, less than 500 nM, less than 100 nM, less than 30 nM, or less than 10 nM. In some embodiments, the apelin receptor agonist has an ERK activation assay EC50 value of less than 20 nM, less than 10 nM, less than 5 nM, less than 2 nM, or less than 1 nM.

In one aspect, the present invention provides, among other things, a method of treating a disease comprising administering the apelin receptor agonist of the present invention to a subject in need thereof. In some embodiments, the disease is pulmonary arterial hypertension (PAH), systemic sclerosis (SSc), heart failure with preserved ejection fraction (HfpEf), age-associated sarcopenia, or acute kidney injury (AKI).

In some embodiments, the present disclosure encompasses a nucleic acid encoding the apelin receptor agonist disclosed herein. In some embodiments, the present disclosure encompasses a method of producing the apelin receptor agonist disclosed herein.

### BRIEF DESCRIPTION OF DRAWINGS

FIG. 1 is an exemplary schematic depiction of how the apelin receptor activation leads to a broad range of physiological actions that are mediated by several signaling pathway.

FIG. 2 is an exemplary graph showing the internalization scores for exemplary apelin analogues against the EC50 values for beta-arrestin signaling and cAMP signaling.

FIG. 3 is an exemplary graph showing the internalization scores for exemplary elabela analogues against the EC50 values for beta-arrestin signaling and cAMP signaling.

### DEFINITIONS

In order for the present disclosure to be more readily understood, certain terms are first defined below. Additional definitions for the following terms and other terms are set forth throughout the specification. The publications and other reference materials referenced herein to describe the background of the disclosure and to provide additional detail regarding its practice are hereby incorporated by reference.

Apelin receptor agonists—The term “apelin receptor agonist” refers to a non-naturally-occurring peptide or polypeptide with an improved agonistic activity on the apelin receptor. In some embodiments, an apelin receptor analogue has an enhanced binding to the apelin receptor as compared to a wild-type apelin. In some embodiments, an apelin receptor analogue has an improved apelin receptor signaling as compared to a wild-type apelin. In some embodiments, an apelin receptor analogue has an increased half-life as compared to a wild-type apelin. In some embodiments, an apelin receptor analogue is more resistant to proteolytic degradation as compared to a wild-type apelin. In some embodiments, an apelin receptor analogue comprises a carrier and a wild-type apelin. In some embodiments, an apelin receptor analogue comprises a carrier and a wild-type elabela.

Fc—The term “Fc” refers to a portion of a heavy chain constant region that comprises at least the CH2 and CH3 domains that typically bind to an Fc receptor e.g., an FcγR, namely FcγRI (CD64), FcγRII (CD32), FcγRIII (CD16) or an FcRn, i.e., a neonatal Fc receptor. The Fc variants of the present invention may be optimized for a variety of properties. An Fc variant that is engineered or predicted to display one or more optimized properties is herein referred to as an “optimized Fc variant”. In some embodiments, an optimized Fc variant has reduced or ablated affinity for FcγRI, FcγRIIa, FcγRIIb, FcγRIIIa, FcγRIIIb, and C1q, and retains binding to FcRn.

Increased half-life—As used herein, “increased half-life” or “increase serum half-life” or “extending half-life” means the positive change in the circulating half-life of a modified biologically active molecule (e.g., apelin or elabela analogue) relative to its non-modified form (or naked form of the peptide). Serum half-life is measured, for example, by taking blood samples at various time points after administration of the biologically active molecule and determining the concentration of that molecule in each sample.

Pharmaceutically Acceptable—As used herein “Pharmaceutically acceptable” refers to being generally recognized for use in animals, and more particularly in humans.

Pharmaceutically Acceptable excipient—As used herein “Pharmaceutically acceptable excipient,” “pharmaceutically acceptable carrier,” or “pharmaceutically acceptable adjuvant” refer, respectively, to an excipient, carrier or adjuvant with which at least one compound of the present disclosure is administered. “Pharmaceutically acceptable vehicle” refers to a diluent, adjuvant, excipient, or carrier with which at least one compound of the present disclosure is administered.

Subject—As used herein “Subject” includes mammals and humans. The terms “human” and “subject” are used interchangeably herein.

Therapeutically effective amount—As used herein “Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

Treating—As used herein “Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

Variant—As used herein, “variant,” “analog” and “mutein” refer to biologically active derivatives of the reference molecule that retain desired activity, such as apelin activity for use in the treatment of a cardiovascular or pulmonary disease or disorder as described herein. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity, and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same apelin biological activity as the native molecule. Methods for making polypeptide analogs and muteins are known in the art and are described further below.

### DETAILED DESCRIPTION

### Apelin Receptor Pathway

The apelin system is an endogenous physiological regulator that is emerging as a potential therapeutic target for many diseases. This system comprises the apelin receptor and its two families of endogenous ligands, apelin and elabela/toddler (ELA; also known as apelin receptor early endogenous ligand).

The APLNR gene (also known as APJ) was identified and cloned in 1993. This gene was found to encode the apelin receptor, a novel G protein-coupled receptor (GPCR) that has ˜50% homology with the type 1 angiotensin II (AT1) receptor. The apelin receptor system opposes the actions of angiotensin II (Ang II) agonism at the AT1 receptor in vitro and in vivo. In the cardiovascular system, apelin and AT1 receptors are co-expressed. The apelin receptor is highly conserved between species with ˜90% sequence similarity between mouse, rat, and human proteins.

### Apelin

Apelin peptides are produced by C-terminal cleavage of a 77-amino-acid precursor, pre-pro-apelin. Peptide fragments of varying lengths circulate in vivo; the major isoforms are apelin-36, apelin-17 and apelin-13. The pyroglutamated form of apelin-13, [Pyr1]apelin-13, is structurally more resistant to metabolism by aminopeptidases than apelin-13 and is the most abundant apelin isoform in the cardiovascular system and human plasma.

The mechanisms of apelin peptide metabolism are not yet fully defined. Experimental evidence suggests that furin (also known as PCSK3), plasma kallikrein and neprilysin cleave apelin peptides, but only neprilysin has been shown to completely inactivate these peptides. The carboxypeptidase angiotensin-converting enzyme 2 (ACE2) has been clearly demonstrated to cleave apelin isoforms both in vitro and in vivo, resulting in the removal of the common C-terminal phenylalanine. This enzyme is highly expressed on the surface of lung alveolar epithelial cells and enterocytes and is expressed at lower levels in most organs including kidney and heart and on arterial and venous endothelial cells.

### Elabela

ELA is a 54-amino-acid peptide that is predicted to be processed to form mature peptides including ELA-32, ELA-21 and ELA-11. The discovery of ELA as the second endogenous ligand for the apelin receptor provided an explanation for the unexpected and marked discrepancy between the phenotypes of apelin receptor-knockout mice, which were not born in expected Mendelian ratios and had substantial cardiovascular developmental defects, and apelin-knockout mice, which developed normally. ELA is detectable in human plasma, but its specific tissue isoform expression has not yet been explored.

### Expression of the Apelin System

Apelin and the apelin receptor are widely expressed within the central nervous system and peripheral organs. In rodents and humans, apelin receptor mRNA is detected in many tissues, particularly in brain, spinal cord, placenta, lung, heart, kidney, adipose tissue, and skeletal muscle. Apelin receptor protein has been identified in the brain, spinal cord, heart, kidney, and lung of rats and humans. In human heart, apelin receptor is expressed by cardiomyocytes, endothelial cells of the endocardium and intramyocardial blood vessel, endothelial cells and smooth muscle of conduit artery and vein. Within the kidney, apelin receptor protein localizes to the cortex and vasculature.

### Apelin Receptor Binding and Signaling

The endogenous apelin isoforms bind with nanomolar affinity to their cognate receptor in cell expression systems and in rat and human cardiovascular tissues. However, structure-activity studies suggest that the longer isoforms, such as apelin-36, and particularly apelin-17, have somewhat higher affinity than the shorter isoforms such as [Pyr¹]apelin-13. The N-terminal RPRL sequence of [Pyr¹]apelin-13, which is present in all longer apelin isoforms, is required for receptor binding with some contribution from other residues such as Pro12. The smallest apelin fragment to retain biological activity, although with reduced affinity, is the 10-amino-acid apelin-13₍₂₋₁₁₎.

A crystal structure has been reported for the apelin receptor in complex with a 17-amino-acid non-endogenous apelin agonist. Substantial mutation was required for successful crystallization of the receptor. However, using this structure in combination with molecular dynamic simulations and molecular modelling, the study demonstrated a two-site peptide-ligand binding mode. This ‘message-address’ concept of peptide binding was also suggested by earlier studies, including one in which amino acids in the N-terminal tail and first extracellular loop were shown to be important both for folding of the receptor protein and binding of apelin peptides. In another structural study, the upper face of the apelin receptor binding site was visualized using 3D homology models, and the interactions of basic residues in the N-terminal RPRL of [Pyr¹]apelin-13 with acidic residues in extracellular loops I and II as well as the interface between extracellular loop III and transmembrane VII were confirmed using site-directed mutagenesis.

The ELA peptides show little sequence similarity to apelin although the predicted isoforms ELA-32, ELA-21 and ELA-11 bind to the apelin receptor in human heart; ELA-32 bound with subnanomolar affinity, ELA-21 with nanomolar affinity (comparable to that of [Pyr¹]apelin-13) and the shortest isoform, ELA-11, bound with affinity an order of magnitude lower than that of the other peptides. An alanine scan and mutagenesis analysis suggested that apelin and ELA peptides engage with different residues in rat and human apelin receptors.

The apelin receptor couples to pertussis toxin-sensitive inhibitory G proteins (Gα_{i/o}) with subsequent activation of extracellular-regulated kinases (ERKs) and phosphoinositide 3-kinase (PI3K)—AKT (also known as protein kinase B (PKB)) signaling cascades. These cascades lead to a broad range of physiological effects that are dependent on the type of cell that is activated. All putative apelin and ELA isoforms elicit canonical Gᵢ-mediated inhibition of adenylate cyclase, which results in inhibition of cAMP. Similar to other GPCRs, the apelin receptor can engage with other heterotrimeric G proteins, particularly G_{q}, resulting in downstream stimulation of phospholipase C (PLC) and AMP-activated protein kinase (AMPK) pathways.

Following activation, GPCRs can be uncoupled from their G proteins and internalized via recruitment of β-arrestins to the receptor. The extent and kinetics of apelin receptor-mediated internalization may be isoform specific, at least in vitro, with apelin-36 suggested to produce more prolonged internalization than [Pyr1]apelin-13. For successful translation of apelin receptor agonists to the clinical setting, the finding that GPCR ligands may preferentially activate a subset of the signaling repertoire of a GPCR or indeed stimulate some signaling pathways and inhibit others, so called ‘biased signaling’, is an important discovery. Compared with [Pyr1]apelin-13, longer apelin ligand isoforms such as apelin-36, apelin-17, ELA-32 and ELA-21 have been shown to exhibit some bias towards β-arrestin recruitment. The in vivo relevance of these observations to apelin receptor physiology or pathophysiology remains to be determined.

Ligand-independent signaling through the apelin receptor has also been reported and may be pathologically important. Phosphorylation of ERK1 and ERK2 downstream of Ang II-mediated activation of AT1 receptor was inhibited in cells that co-expressed the apelin receptor and this inhibition was abolished by apelin in a pertussis toxin-sensitive manner. Subsequently, genetic loss of the apelin receptor was demonstrated to protect against cardiac hypertrophy and heart failure in a chronic pressure overload mouse model, whereas loss of apelin had no effect. This discrepancy could potentially be explained by the later discovery of ELA. However, isolated cardiomyocytes from apelin receptor-knockout mice exhibited a reduced response to stretch, implying that the apelin receptor can act as a mechanosensor even in the absence of ligand. This stretch-mediated response was abrogated by knockdown of β-arrestins and blunted by the addition of apelin. The apelin receptor might therefore have pro-hypertrophic effects via β-arrestin signaling in the absence of ligand and anti-hypertrophic effects via (3-arrestin-independent pathways when activated by apelin.

### Apelin Receptor Agonists

### Apelin Analogues

The present invention provides, among other things, an apelin analogue with an improved agonistic activity on the apelin receptor. In some embodiments, an apelin analogue has an enhanced binding to the apelin receptor as compared to a wild-type apelin. In some embodiments, an apelin analogue has an improved apelin receptor signaling as compared to a wild-type apelin. In some embodiments, an apelin analogue has an increased half-life as compared to a wild-type apelin. In some embodiments, an apelin analog is more resistant to proteolytic degradation as compared to a wild-type apelin.

In some embodiments, an apelin analogue is derived from wild-type apelin-77 (MNLRLCVQALLLLWLSLTAVCGGSLMPLPDGNGLEDGNVRHLVQPRGSRNGPGPWQ GGRRKFRRQRPRLSHKGPMPF) of SEQ ID NO: 42.

In some embodiments, an apelin analogue is derived from wild-type apelin-36 (LVQPRGSRNGPGPWQGGRRKFRRQRPRLSHKGPMPF) of SEQ ID NO: 43. In some embodiments, an apelin analogue is derived from wild-type apelin-17 (KFRRQRPRLSHKGPMPF) of SEQ ID NO: 44. In some embodiments, an apelin analogue is derived from wild-type apelin-13 (QRPRLSHKGPMPF) of SEQ ID NO: 1. In some embodiments, an apelin analogue is derived from [pyr-Glu]apelin-13 (ERPRLSHKGPMPF) of SEQ ID NO: 45.

In some embodiments, an apelin analog comprises a modification as compared to SEQ ID NO: 1, SEQ ID NO: 42, SEQ ID NO: 43, SEQ ID NO: 44, or SEQ ID NO:45. In some embodiments, a modification is a substitution, an insertion, or a deletion of one or more amino acids.

In some embodiments, an apelin analogue comprises one or more amino acid substitutions at position 1, 2, 3, 4, 5, 6, 7, 8, 9, 10, 11, 12 or 13 of SEQ ID NO: 1. In some embodiments, an apelin analogue comprises one or more amino acid substitutions at position 1, 6, 8, 9, 11, 12, or 13 of SEQ ID NO: 1. In some embodiments, an apelin analogue comprises an amino acid substitutions at position 12 of SEQ ID NO: 1. In some embodiments, an apelin analogue comprises an amino acid substitutions at position 8 of SEQ ID NO: 1. In some embodiments, an apelin analogue comprises an amino acid substitutions at position 1 of SEQ ID NO: 1. In some embodiments, an apelin analogue comprises an amino acid substitutions at position 11 of SEQ ID NO: 1. In some embodiments, an apelin analogue comprises an amino acid substitutions at position 13 of SEQ ID NO: 1. In some embodiments, an apelin analogue comprises amino acid substitutions at positions 11 and 13 of SEQ ID NO: 1. In some embodiments, an apelin analogue comprises amino acid substitutions at positions 12 and 13 of SEQ ID NO: 1. In some embodiments, an apelin analogue comprises amino acid substitutions at positions 11, 12 and 13 of SEQ ID NO: 1. In some embodiments, an apelin analogue comprises amino acid substitutions at positions 8, 9, and 12 of SEQ ID NO: 1. In some embodiments, an apelin analogue comprises amino acid substitutions at positions 8, 9, 12 and 13 of SEQ ID NO: 1. In some embodiments, an apelin analogue comprises amino acid substitutions at positions 8, 12, and 13 of SEQ ID NO: 1.

In some embodiments, an apelin analogue comprises P12V amino acid substitution as compared to SEQ ID NO: 1. In some embodiments, an apelin analogue comprises M11H amino acid substitution as compared to SEQ ID NO: 1. In some embodiments, an apelin analogue comprises K8W amino acid substitution as compared to SEQ ID NO: 1. In some embodiments, an apelin analogue comprises Q1E amino acid substitution as compared to SEQ ID NO: 1. In some embodiments, an apelin analogue comprises M11A and F13Y amino acid substitutions as compared to SEQ ID NO: 1. In some embodiments, an apelin analogue comprises P12A amino acid substitution as compared to SEQ ID NO: 1. In some embodiments, an apelin analogue comprises S6Q amino acid substitution as compared to SEQ ID NO: 1. In some embodiments, an apelin analogue comprises G9N amino acid substitution as compared to SEQ ID NO: 1. In some embodiments, an apelin analogue comprises P12H amino acid substitution as compared to SEQ ID NO: 1. In some embodiments, an apelin analogue comprises F13W amino acid substitution as compared to SEQ ID NO: 1. In some embodiments, an apelin analogue comprises P12H and F13W amino acid substitutions as compared to SEQ ID NO: 1. In some embodiments, an apelin analogue comprises M11A, P12A and F13W amino acid substitutions as compared to SEQ ID NO: 1. In some embodiments, an apelin analogue comprises M11A, P12A and F13Y amino acid substitutions as compared to SEQ ID NO: 1. In some embodiments, an apelin analogue comprises S6Q, G9N and P12A amino acid substitutions as compared to SEQ ID NO: 1. In some embodiments, an apelin analogue comprises S6Q, G9N, P12A and F13W amino acid substitutions as compared to SEQ ID NO: 1. In some embodiments, an apelin analogue comprises K8W, P12A, and F13W amino acid substitutions as compared to SEQ ID NO: 1.

In some embodiments, an apelin analogue comprises amino acid sequence shown in Table 1.

TABLE 1
Exemplary Apelin Analogues

| | | SEQ ID |
|---|---|---|
| Sequence | Modification | NO: |
| QRPRLSHKGPMPF | | 1 |
| QRPRLSHKGPMVF | P12V | 2 |
| QRPRLSHWGPMPF | K8W | 3 |
| ERPRLSHKGPMPF | Q1E | 4 |
| QRPRLSHKGPHPF | M11H | 5 |
| QRPRLSHKGPAPY | M11A, F13Y | 6 |
| QRPRLSHKGPMAF | P12A | 7 |
| QRPRLSHKGPMHW | P12H, F13W | 50 |
| QRPRLSHKGPAAW | M11A, P12A, F13W | 51 |
| QRPRLSHKGPAAY | M11A, P12A, F13Y | 52 |
| QRPRLQHKNPMAF | S6Q, G9N, P12A | 53 |
| QRPRLQHKNPMAW | S6Q, G9N, P12A, F13W | 54 |
| QRPRLSHWGPMAW | K8W, P12A, F13W | 55 |

In some embodiments, an apelin analogue comprises the amino acid sequence of SEQ ID NO: 1. In some embodiments, an apelin analogue comprises the amino acid sequence of SEQ ID NO: 2. In some embodiments, an apelin analogue comprises the amino acid sequence of SEQ ID NO: 3. In some embodiments, an apelin analogue comprises the amino acid sequence of SEQ ID NO: 4. In some embodiments, an apelin analogue comprises the amino acid sequence of SEQ ID NO: 5. In some embodiments, an apelin analogue comprises the amino acid sequence of SEQ ID NO: 6. In some embodiments, an apelin analogue comprises the amino acid sequence of SEQ ID NO: 7. In some embodiments, an apelin analogue comprises the amino acid sequence of SEQ ID NO: 50. In some embodiments, an apelin analogue comprises the amino acid sequence of SEQ ID NO: 51. In some embodiments, an apelin analogue comprises the amino acid sequence of SEQ ID NO: 52. In some embodiments, an apelin analogue comprises the amino acid sequence of SEQ ID NO: 53. In some embodiments, an apelin analogue comprises the amino acid sequence of SEQ ID NO: 54. In some embodiments, an apelin analogue comprises the amino acid sequence of SEQ ID NO: 55.

In some embodiments, an apelin analogue differs no more than 4 amino acid residues from SEQ ID NO: 1. In some embodiments, an apelin analogue differs no more than 3 amino acid residues from SEQ ID NO: 1. In some embodiments, an apelin analogue differs no more than 2 amino acid residues from SEQ ID NO: 1. In some embodiments, an apelin analogue differs no more than 1 amino acid residue from SEQ ID NO: 1.

In some embodiments, an apelin analogue differs no more than 3 amino acid residues from SEQ ID NO: 2. In some embodiments, an apelin analogue differs no more than 2 amino acid residues from SEQ ID NO: 2. In some embodiments, an apelin analogue differs no more than 1 amino acid residue from SEQ ID NO: 2.

In some embodiments, an apelin analogue differs no more than 3 amino acid residues from SEQ ID NO: 3. In some embodiments, an apelin analogue differs no more than 2 amino acid residues from SEQ ID NO: 3. In some embodiments, an apelin analogue differs no more than 1 amino acid residue from SEQ ID NO: 3.

In some embodiments, an apelin analogue differs no more than 3 amino acid residues from SEQ ID NO: 4. In some embodiments, an apelin analogue differs no more than 2 amino acid residues from SEQ ID NO: 4. In some embodiments, an apelin analogue differs no more than 1 amino acid residue from SEQ ID NO: 4.

In some embodiments, an apelin analogue differs no more than 3 amino acid residues from SEQ ID NO: 5. In some embodiments, an apelin analogue differs no more than 2 amino acid residues from SEQ ID NO: 5. In some embodiments, an apelin analogue differs no more than 1 amino acid residue from SEQ ID NO: 5.

In some embodiments, an apelin analogue differs no more than 3 amino acid residues from SEQ ID NO: 6. In some embodiments, an apelin analogue differs no more than 2 amino acid residues from SEQ ID NO: 6. In some embodiments, an apelin analogue differs no more than 1 amino acid residue from SEQ ID NO: 6.

In some embodiments, an apelin analogue differs no more than 3 amino acid residues from SEQ ID NO: 7. In some embodiments, an apelin analogue differs no more than 2 amino acid residues from SEQ ID NO: 7. In some embodiments, an apelin analogue differs no more than 1 amino acid residue from SEQ ID NO: 7.

In some embodiments, an apelin analogue differs no more than 3 amino acid residues from SEQ ID NO: 50. In some embodiments, an apelin analogue differs no more than 2 amino acid residues from SEQ ID NO: 50. In some embodiments, an apelin analogue differs no more than 1 amino acid residue from SEQ ID NO: 50.

In some embodiments, an apelin analogue differs no more than 3 amino acid residues from SEQ ID NO: 51. In some embodiments, an apelin analogue differs no more than 2 amino acid residues from SEQ ID NO: 51. In some embodiments, an apelin analogue differs no more than 1 amino acid residue from SEQ ID NO: 51.

In some embodiments, an apelin analogue differs no more than 3 amino acid residues from SEQ ID NO: 52. In some embodiments, an apelin analogue differs no more than 2 amino acid residues from SEQ ID NO: 52. In some embodiments, an apelin analogue differs no more than 1 amino acid residue from SEQ ID NO: 52.

In some embodiments, an apelin analogue differs no more than 3 amino acid residues from SEQ ID NO: 53. In some embodiments, an apelin analogue differs no more than 2 amino acid residues from SEQ ID NO: 53. In some embodiments, an apelin analogue differs no more than 1 amino acid residue from SEQ ID NO: 53.

In some embodiments, an apelin analogue differs no more than 3 amino acid residues from SEQ ID NO: 54. In some embodiments, an apelin analogue differs no more than 2 amino acid residues from SEQ ID NO: 54. In some embodiments, an apelin analogue differs no more than 1 amino acid residue from SEQ ID NO: 54.

In some embodiments, an apelin analogue differs no more than 3 amino acid residues from SEQ ID NO: 55. In some embodiments, an apelin analogue differs no more than 2 amino acid residues from SEQ ID NO: 55. In some embodiments, an apelin analogue differs no more than 1 amino acid residue from SEQ ID NO: 55.

In some embodiments, an apelin analogue comprises sequence that is at least 90% identical to SEQ ID NO: 1. In some embodiments, an apelin analogue comprises sequence that is at least 92% identical to SEQ ID NO: 1. In some embodiments, an apelin analogue comprises sequence that is at least 95% identical to SEQ ID NO: 1. In some embodiments, an apelin analogue comprises sequence that is at least 90% identical to SEQ ID NO: 1. In some embodiments, an apelin analogue comprises sequence that is at least 96% identical to SEQ ID NO: 1. In some embodiments, an apelin analogue comprises sequence that is at least 97% identical to SEQ ID NO: 1. In some embodiments, an apelin analogue comprises sequence that is at least 98% identical to SEQ ID NO: 1. In some embodiments, an apelin analogue comprises sequence that is at least 99% identical to SEQ ID NO: 1.

In some embodiments, an apelin analogue comprises sequence that is at least 90% identical to SEQ ID NO: 2. In some embodiments, an apelin analogue comprises sequence that is at least 92% identical to SEQ ID NO: 2. In some embodiments, an apelin analogue comprises sequence that is at least 95% identical to SEQ ID NO: 2. In some embodiments, an apelin analogue comprises sequence that is at least 90% identical to SEQ ID NO: 2. In some embodiments, an apelin analogue comprises sequence that is at least 96% identical to SEQ ID NO: 2. In some embodiments, an apelin analogue comprises sequence that is at least 97% identical to SEQ ID NO: 2. In some embodiments, an apelin analogue comprises sequence that is at least 98% identical to SEQ ID NO: 2. In some embodiments, an apelin analogue comprises sequence that is at least 99% identical to SEQ ID NO: 2.

In some embodiments, an apelin analogue comprises sequence that is at least 90% identical to SEQ ID NO: 3. In some embodiments, an apelin analogue comprises sequence that is at least 92% identical to SEQ ID NO: 3. In some embodiments, an apelin analogue comprises sequence that is at least 95% identical to SEQ ID NO: 3. In some embodiments, an apelin analogue comprises sequence that is at least 90% identical to SEQ ID NO: 3. In some embodiments, an apelin analogue comprises sequence that is at least 96% identical to SEQ ID NO: 3. In some embodiments, an apelin analogue comprises sequence that is at least 97% identical to SEQ ID NO: 3. In some embodiments, an apelin analogue comprises sequence that is at least 98% identical to SEQ ID NO: 3. In some embodiments, an apelin analogue comprises sequence that is at least 99% identical to SEQ ID NO: 3.

In some embodiments, an apelin analogue comprises sequence that is at least 90% identical to SEQ ID NO: 4. In some embodiments, an apelin analogue comprises sequence that is at least 92% identical to SEQ ID NO: 4. In some embodiments, an apelin analogue comprises sequence that is at least 95% identical to SEQ ID NO: 4. In some embodiments, an apelin analogue comprises sequence that is at least 90% identical to SEQ ID NO: 4. In some embodiments, an apelin analogue comprises sequence that is at least 96% identical to SEQ ID NO: 4. In some embodiments, an apelin analogue comprises sequence that is at least 97% identical to SEQ ID NO: 4. In some embodiments, an apelin analogue comprises sequence that is at least 98% identical to SEQ ID NO: 4. In some embodiments, an apelin analogue comprises sequence that is at least 99% identical to SEQ ID NO: 4.

In some embodiments, an apelin analogue comprises sequence that is at least 90% identical to SEQ ID NO: 5. In some embodiments, an apelin analogue comprises sequence that is at least 92% identical to SEQ ID NO: 5. In some embodiments, an apelin analogue comprises sequence that is at least 95% identical to SEQ ID NO: 5. In some embodiments, an apelin analogue comprises sequence that is at least 90% identical to SEQ ID NO: 5. In some embodiments, an apelin analogue comprises sequence that is at least 96% identical to SEQ ID NO: 5. In some embodiments, an apelin analogue comprises sequence that is at least 97% identical to SEQ ID NO: 5. In some embodiments, an apelin analogue comprises sequence that is at least 98% identical to SEQ ID NO: 5. In some embodiments, an apelin analogue comprises sequence that is at least 99% identical to SEQ ID NO: 5.

In some embodiments, an apelin analogue comprises sequence that is at least 90% identical to SEQ ID NO: 6. In some embodiments, an apelin analogue comprises sequence that is at least 92% identical to SEQ ID NO: 6. In some embodiments, an apelin analogue comprises sequence that is at least 95% identical to SEQ ID NO: 6. In some embodiments, an apelin analogue comprises sequence that is at least 90% identical to SEQ ID NO: 6. In some embodiments, an apelin analogue comprises sequence that is at least 96% identical to SEQ ID NO: 6. In some embodiments, an apelin analogue comprises sequence that is at least 97% identical to SEQ ID NO: 6. In some embodiments, an apelin analogue comprises sequence that is at least 98% identical to SEQ ID NO: 6. In some embodiments, an apelin analogue comprises sequence that is at least 99% identical to SEQ ID NO: 6.

In some embodiments, an apelin analogue comprises sequence that is at least 90% identical to SEQ ID NO: 7. In some embodiments, an apelin analogue comprises sequence that is at least 92% identical to SEQ ID NO: 7. In some embodiments, an apelin analogue comprises sequence that is at least 95% identical to SEQ ID NO: 7. In some embodiments, an apelin analogue comprises sequence that is at least 90% identical to SEQ ID NO: 7. In some embodiments, an apelin analogue comprises sequence that is at least 96% identical to SEQ ID NO: 7. In some embodiments, an apelin analogue comprises sequence that is at least 97% identical to SEQ ID NO: 7. In some embodiments, an apelin analogue comprises sequence that is at least 98% identical to SEQ ID NO: 7. In some embodiments, an apelin analogue comprises sequence that is at least 99% identical to SEQ ID NO: 7.

In some embodiments, an apelin analogue comprises sequence that is at least 90% identical to SEQ ID NO: 50. In some embodiments, an apelin analogue comprises sequence that is at least 92% identical to SEQ ID NO: 50. In some embodiments, an apelin analogue comprises sequence that is at least 95% identical to SEQ ID NO: 50. In some embodiments, an apelin analogue comprises sequence that is at least 90% identical to SEQ ID NO: 50. In some embodiments, an apelin analogue comprises sequence that is at least 96% identical to SEQ ID NO: 50. In some embodiments, an apelin analogue comprises sequence that is at least 97% identical to SEQ ID NO: 50. In some embodiments, an apelin analogue comprises sequence that is at least 98% identical to SEQ ID NO: 50. In some embodiments, an apelin analogue comprises sequence that is at least 99% identical to SEQ ID NO: 50.

In some embodiments, an apelin analogue comprises sequence that is at least 90% identical to SEQ ID NO: 51. In some embodiments, an apelin analogue comprises sequence that is at least 92% identical to SEQ ID NO: 51. In some embodiments, an apelin analogue comprises sequence that is at least 95% identical to SEQ ID NO: 51. In some embodiments, an apelin analogue comprises sequence that is at least 90% identical to SEQ ID NO: 51. In some embodiments, an apelin analogue comprises sequence that is at least 96% identical to SEQ ID NO: 51. In some embodiments, an apelin analogue comprises sequence that is at least 97% identical to SEQ ID NO: 51. In some embodiments, an apelin analogue comprises sequence that is at least 98% identical to SEQ ID NO: 51. In some embodiments, an apelin analogue comprises sequence that is at least 99% identical to SEQ ID NO: 51.

In some embodiments, an apelin analogue comprises sequence that is at least 90% identical to SEQ ID NO: 52. In some embodiments, an apelin analogue comprises sequence that is at least 92% identical to SEQ ID NO: 52. In some embodiments, an apelin analogue comprises sequence that is at least 95% identical to SEQ ID NO: 52. In some embodiments, an apelin analogue comprises sequence that is at least 90% identical to SEQ ID NO: 52. In some embodiments, an apelin analogue comprises sequence that is at least 96% identical to SEQ ID NO: 52. In some embodiments, an apelin analogue comprises sequence that is at least 97% identical to SEQ ID NO: 52. In some embodiments, an apelin analogue comprises sequence that is at least 98% identical to SEQ ID NO: 52. In some embodiments, an apelin analogue comprises sequence that is at least 99% identical to SEQ ID NO: 52.

In some embodiments, an apelin analogue comprises sequence that is at least 90% identical to SEQ ID NO: 53. In some embodiments, an apelin analogue comprises sequence that is at least 92% identical to SEQ ID NO: 53. In some embodiments, an apelin analogue comprises sequence that is at least 95% identical to SEQ ID NO: 53. In some embodiments, an apelin analogue comprises sequence that is at least 90% identical to SEQ ID NO: 53. In some embodiments, an apelin analogue comprises sequence that is at least 96% identical to SEQ ID NO: 53. In some embodiments, an apelin analogue comprises sequence that is at least 97% identical to SEQ ID NO: 53. In some embodiments, an apelin analogue comprises sequence that is at least 98% identical to SEQ ID NO: 53. In some embodiments, an apelin analogue comprises sequence that is at least 99% identical to SEQ ID NO: 53.

In some embodiments, an apelin analogue comprises sequence that is at least 90% identical to SEQ ID NO: 54. In some embodiments, an apelin analogue comprises sequence that is at least 92% identical to SEQ ID NO: 54. In some embodiments, an apelin analogue comprises sequence that is at least 95% identical to SEQ ID NO: 54. In some embodiments, an apelin analogue comprises sequence that is at least 90% identical to SEQ ID NO: 54. In some embodiments, an apelin analogue comprises sequence that is at least 96% identical to SEQ ID NO: 54. In some embodiments, an apelin analogue comprises sequence that is at least 97% identical to SEQ ID NO: 54. In some embodiments, an apelin analogue comprises sequence that is at least 98% identical to SEQ ID NO: 54. In some embodiments, an apelin analogue comprises sequence that is at least 99% identical to SEQ ID NO: 54.

In some embodiments, an apelin analogue comprises sequence that is at least 90% identical to SEQ ID NO: 55. In some embodiments, an apelin analogue comprises sequence that is at least 92% identical to SEQ ID NO: 55. In some embodiments, an apelin analogue comprises sequence that is at least 95% identical to SEQ ID NO: 55. In some embodiments, an apelin analogue comprises sequence that is at least 90% identical to SEQ ID NO: 55. In some embodiments, an apelin analogue comprises sequence that is at least 96% identical to SEQ ID NO: 55. In some embodiments, an apelin analogue comprises sequence that is at least 97% identical to SEQ ID NO: 55. In some embodiments, an apelin analogue comprises sequence that is at least 98% identical to SEQ ID NO: 55. In some embodiments, an apelin analogue comprises sequence that is at least 99% identical to SEQ ID NO: 55.

In some embodiments, an apelin analogue comprises the amino acid sequence

(SEQ ID NO: 63)
X1-R-P-R-L-S-H-X2-G-P-X3-X4-X5

wherein
X is Q or E;
X2 is K or W;
X3 is M, H, or A;
X4 is P, V, A, or H; and/or
X5 is F, Y, or W.

In some embodiments, an apelin analogue comprises the amino acid sequence

(SEQ ID NO: 64)
Q-R-P-R-L-Q-H-K-N-P-M-A-X1

wherein
X1 is F or W.

In some embodiments, an apelin analogue comprises the amino acid sequence

(SEQ ID NO: 65)
Q-R-P-R-L-S-H-K-G-P-X1-X2-X3

wherein
X1 is M, H, or A;
X2 is P, V, A, or H; and/or
X3 is F, Y, or W.

In some embodiments, an apelin analogue comprises the amino acid sequence

(SEQ ID NO: 66)
X1-R-P-R-L-X2-H-X3-X4-P-X5-X6-X7

wherein
X1 is Q or E;
X2 is S or Q;
X3 is K or W;
X4 is G or N;
X5 is M, H, or A;
X6 is P, V, A or H; and/or
X7 is F, Y or W.

### Elabela Analogues

The present invention provides, among other things, an elabela analogue with an improved agonistic activity on the apelin receptor. In some embodiments, an elabela analogue has an enhanced binding to the apelin receptor as compared to a wild-type elabela. In some embodiments, an elabela analogue has an improved apelin receptor signaling as compared to a wild-type elabela. In some embodiments, an elabela analogue has an increased half-life as compared to a wild-type elabela. In some embodiments, an apelin analog is more resistant to proteolytic degradation as compared to a wild-type elabela.

In some embodiments, an elabela analogue is derived from wild-type elabela-54 (MRFQQFLFAFFIFIMSLLLISGQRPVNLTMRRKLRKHNCLQRRCMPLHSRVPFP) of SEQ ID NO: 46. In some embodiments, an elabela analogue is derived from wild-type elabela-32 (QRPVNLTMRRKLRKHNCLQRRCMPLHSRVPFP) of SEQ ID NO: 47. In some embodiments, an elabela analogue is derived from wild-type elabela-22 (KLRKHNCLQRRCMPLHSRVPFP) of SEQ ID NO: 8. In some embodiments, an elabela analogue is derived from wild-type elabela-21 (LRKHNCLQRRCMPLHSRVPFP) of SEQ ID NO: 48. In some embodiments, an elabela analogue is derived from wild-type elabela-11 (CMPLHSRVPFP) of SEQ ID NO: 49.

In some embodiments, an elabela analog comprises a modification as compared to SEQ ID NO: 8, SEQ ID NO: 46, SEQ ID NO: 47, SEQ ID NO: 48, or SEQ ID NO:49. In some embodiments, a modification is a substitution, an insertion, or a deletion of one or more amino acids.

In some embodiments, an elabela analogue comprises one or more amino acid substitutions at position 1, 2, 3, 4, 5, 6, 7, 8, 9, 10, 11, 12, 13, 14, 15, 16, 17, 18, 19, 20, 21, or 22 of SEQ ID NO: 8. In some embodiments, an elabela analogue comprises one or more amino acid substitutions at position 3, 7, 8, 9, 10, 12, 14, 15, 17, or 18 of SEQ ID NO: 8. In some embodiments, an elabela analogue comprises an amino acid substitutions at position 3 of SEQ ID NO: 8. In some embodiments, an elabela analogue comprises an amino acid substitutions at position 7 of SEQ ID NO: 8. In some embodiments, an elabela analogue comprises an amino acid substitutions at position 8 of SEQ ID NO: 8. In some embodiments, an elabela analogue comprises an amino acid substitutions at position 9 of SEQ ID NO: 8. In some embodiments, an elabela analogue comprises an amino acid substitutions at position 10 of SEQ ID NO: 8. In some embodiments, an elabela analogue comprises an amino acid substitutions at position 12 of SEQ ID NO: 8. In some embodiments, an elabela analogue comprises an amino acid substitutions at position 14 of SEQ ID NO: 8. In some embodiments, an elabela analogue comprises an amino acid substitutions at position 15 of SEQ ID NO: 8. In some embodiments, an elabela analogue comprises an amino acid substitutions at position 17 of SEQ ID NO: 8. In some embodiments, an elabela analogue comprises an amino acid substitutions at position 18 of SEQ ID NO: 8.

In some embodiments, an elabela analogue comprises R3N amino acid substitution as compared to SEQ ID NO: 8. In some embodiments, an elabela analogue comprises P14I amino acid substitution as compared to SEQ ID NO: 8. In some embodiments, an elabela analogue comprises S17Q amino acid substitution as compared to SEQ ID NO: 8. In some embodiments, an elabela analogue comprises S17R amino acid substitution as compared to SEQ ID NO: 8. In some embodiments, an elabela analogue comprises Q9K amino acid substitution as compared to SEQ ID NO: 8. In some embodiments, an elabela analogue comprises R18Y amino acid substitution as compared to SEQ ID NO: 8. In some embodiments, an elabela analogue comprises L15Y amino acid substitution as compared to SEQ ID NO: 8. In some embodiments, an elabela analogue comprises C7S and C12Y amino acid substitutions as compared to SEQ ID NO: 8. In some embodiments, an elabela analogue comprises R10E amino acid substitution as compared to SEQ ID NO: 8. In some embodiments, an elabela analogue comprises Q9I amino acid substitution as compared to SEQ ID NO: 8. In some embodiments, an elabela analogue comprises L8A and Q9T amino acid substitution as compared to SEQ ID NO: 8.

In some embodiments, an apelin analogue comprises amino acid sequence shown in Table 2.

TABLE 2
Exemplary Elabela Analogues

| | | SEQ |
|---|---|---|
| | Modifica- | ID |
| Sequence | tion | NO: |
| KLRKHNCLQRRCMPLHSRVPFP | | 8 |
| KLNKHNCLQRRCMPLHSRVPFP | R3N | 9 |
| KLRKHNCLQRRCMILHSRVPFP | P14I | 10 |
| KLRKHNCLQRRCMPLHQRVPFP | S17Q | 11 |
| KLRKHNCLQRRCMPLHRRVPFP | S17R | 12 |
| KLRKHNCLKRRCMPLHSRVPFP | Q9K | 13 |
| KLRKHNCLQRRCMPLHSYVPFP | R18Y | 30 |
| KLRKHNCLQRRCMPYHSRVPFP | L15Y | 31 |
| KLRKHNSLQRRYMPLHSRVPFP | C7S, | 32 |
| | C12Y | |
| KLRKHNCLQERCMPLHSRVPFP | R10E | 33 |
| KLRKHNCLIRRCMPLHSRVPFP | Q9I | 34 |
| KLRKHNCATRRCMPLHSRVPFP | L8A, Q9T | 35 |

In some embodiments, an elabela analogue comprises the amino acid sequence of SEQ ID NO: 8. In some embodiments, an elabela analogue comprises the amino acid sequence of SEQ ID NO: 9. In some embodiments, an elabela analogue comprises the amino acid sequence of SEQ ID NO: 10. In some embodiments, an elabela analogue comprises the amino acid sequence of SEQ ID NO: 11. In some embodiments, an elabela analogue comprises the amino acid sequence of SEQ ID NO: 12. In some embodiments, an elabela analogue comprises the amino acid sequence of SEQ ID NO: 13. In some embodiments, an elabela analogue comprises the amino acid sequence of SEQ ID NO: 30. In some embodiments, an elabela analogue comprises the amino acid sequence of SEQ ID NO: 31. In some embodiments, an elabela analogue comprises the amino acid sequence of SEQ ID NO: 32. In some embodiments, an elabela analogue comprises the amino acid sequence of SEQ ID NO: 33. In some embodiments, an elabela analogue comprises the amino acid sequence of SEQ ID NO: 34. In some embodiments, an elabela analogue comprises the amino acid sequence of SEQ ID NO: 35.

In some embodiments, an elabela analogue differs no more than 5 amino acid residues from SEQ ID NO: 8. In some embodiments, an elabela analogue differs no more than 4 amino acid residues from SEQ ID NO: 8. In some embodiments, an elabela analogue differs no more than 3 amino acid residues from SEQ ID NO: 8. In some embodiments, an elabela analogue differs no more than 2 amino acid residues from SEQ ID NO: 8. In some embodiments, an elabela analogue differs no more than 1 amino acid residue from SEQ ID NO: 8.

In some embodiments, an elabela analogue differs no more than 5 amino acid residues from SEQ ID NO: 9. In some embodiments, an elabela analogue differs no more than 4 amino acid residues from SEQ ID NO: 9. In some embodiments, an elabela analogue differs no more than 3 amino acid residues from SEQ ID NO: 9. In some embodiments, an elabela analogue differs no more than 2 amino acid residues from SEQ ID NO: 9. In some embodiments, an elabela analogue differs no more than 1 amino acid residue from SEQ ID NO: 9.

In some embodiments, an elabela analogue differs no more than 5 amino acid residues from SEQ ID NO: 10. In some embodiments, an elabela analogue differs no more than 4 amino acid residues from SEQ ID NO: 10. In some embodiments, an elabela analogue differs no more than 3 amino acid residues from SEQ ID NO: 10. In some embodiments, an elabela analogue differs no more than 2 amino acid residues from SEQ ID NO: 10. In some embodiments, an elabela analogue differs no more than 1 amino acid residue from SEQ ID NO: 10.

In some embodiments, an elabela analogue differs no more than 5 amino acid residues from SEQ ID NO: 11. In some embodiments, an elabela analogue differs no more than 4 amino acid residues from SEQ ID NO: 11. In some embodiments, an elabela analogue differs no more than 3 amino acid residues from SEQ ID NO: 11. In some embodiments, an elabela analogue differs no more than 2 amino acid residues from SEQ ID NO: 11. In some embodiments, an elabela analogue differs no more than 1 amino acid residue from SEQ ID NO: 11.

In some embodiments, an elabela analogue differs no more than 5 amino acid residues from SEQ ID NO: 12. In some embodiments, an elabela analogue differs no more than 4 amino acid residues from SEQ ID NO: 12. In some embodiments, an elabela analogue differs no more than 3 amino acid residues from SEQ ID NO: 12. In some embodiments, an elabela analogue differs no more than 2 amino acid residues from SEQ ID NO: 12. In some embodiments, an elabela analogue differs no more than 1 amino acid residue from SEQ ID NO: 12.

In some embodiments, an elabela analogue differs no more than 5 amino acid residues from SEQ ID NO: 13. In some embodiments, an elabela analogue differs no more than 4 amino acid residues from SEQ ID NO: 13. In some embodiments, an elabela analogue differs no more than 3 amino acid residues from SEQ ID NO: 13. In some embodiments, an elabela analogue differs no more than 2 amino acid residues from SEQ ID NO: 3. In some embodiments, an elabela analogue differs no more than 1 amino acid residue from SEQ ID NO: 13.

In some embodiments, an elabela analogue differs no more than 5 amino acid residues from SEQ ID NO: 30. In some embodiments, an elabela analogue differs no more than 4 amino acid residues from SEQ ID NO: 30. In some embodiments, an elabela analogue differs no more than 3 amino acid residues from SEQ ID NO: 30. In some embodiments, an elabela analogue differs no more than 2 amino acid residues from SEQ ID NO: 30. In some embodiments, an elabela analogue differs no more than 1 amino acid residue from SEQ ID NO: 30.

In some embodiments, an elabela analogue differs no more than 5 amino acid residues from SEQ ID NO: 31. In some embodiments, an elabela analogue differs no more than 4 amino acid residues from SEQ ID NO: 31. In some embodiments, an elabela analogue differs no more than 3 amino acid residues from SEQ ID NO: 31. In some embodiments, an elabela analogue differs no more than 2 amino acid residues from SEQ ID NO: 31. In some embodiments, an elabela analogue differs no more than 1 amino acid residue from SEQ ID NO: 31.

In some embodiments, an elabela analogue differs no more than 5 amino acid residues from SEQ ID NO: 32. In some embodiments, an elabela analogue differs no more than 4 amino acid residues from SEQ ID NO: 32. In some embodiments, an elabela analogue differs no more than 3 amino acid residues from SEQ ID NO: 32. In some embodiments, an elabela analogue differs no more than 2 amino acid residues from SEQ ID NO: 32. In some embodiments, an elabela analogue differs no more than 1 amino acid residue from SEQ ID NO: 32.

In some embodiments, an elabela analogue differs no more than 5 amino acid residues from SEQ ID NO: 33. In some embodiments, an elabela analogue differs no more than 4 amino acid residues from SEQ ID NO: 33. In some embodiments, an elabela analogue differs no more than 3 amino acid residues from SEQ ID NO: 33. In some embodiments, an elabela analogue differs no more than 2 amino acid residues from SEQ ID NO: 33. In some embodiments, an elabela analogue differs no more than 1 amino acid residue from SEQ ID NO: 33.

In some embodiments, an elabela analogue differs no more than 5 amino acid residues from SEQ ID NO: 34. In some embodiments, an elabela analogue differs no more than 4 amino acid residues from SEQ ID NO: 34. In some embodiments, an elabela analogue differs no more than 3 amino acid residues from SEQ ID NO: 34. In some embodiments, an elabela analogue differs no more than 2 amino acid residues from SEQ ID NO: 34. In some embodiments, an elabela analogue differs no more than 1 amino acid residue from SEQ ID NO: 34.

In some embodiments, an elabela analogue differs no more than 5 amino acid residues from SEQ ID NO: 35. In some embodiments, an elabela analogue differs no more than 4 amino acid residues from SEQ ID NO: 35. In some embodiments, an elabela analogue differs no more than 3 amino acid residues from SEQ ID NO: 35. In some embodiments, an elabela analogue differs no more than 2 amino acid residues from SEQ ID NO: 35. In some embodiments, an elabela analogue differs no more than 1 amino acid residue from SEQ ID NO: 35.

In some embodiments, an elabela analogue comprises sequence that is at least 90% identical to SEQ ID NO: 8. In some embodiments, an elabela analogue comprises sequence that is at least 92% identical to SEQ ID NO: 8. In some embodiments, an elabela analogue comprises sequence that is at least 95% identical to SEQ ID NO: 8. In some embodiments, an elabela analogue comprises sequence that is at least 90% identical to SEQ ID NO: 8. In some embodiments, an elabela analogue comprises sequence that is at least 96% identical to SEQ ID NO: 8. In some embodiments, an elabela analogue comprises sequence that is at least 97% identical to SEQ ID NO: 8. In some embodiments, an elabela analogue comprises sequence that is at least 98% identical to SEQ ID NO: 8. In some embodiments, an elabela analogue comprises sequence that is at least 99% identical to SEQ ID NO: 8.

In some embodiments, an elabela analogue comprises sequence that is at least 90% identical to SEQ ID NO: 9. In some embodiments, an elabela analogue comprises sequence that is at least 92% identical to SEQ ID NO: 9. In some embodiments, an elabela analogue comprises sequence that is at least 95% identical to SEQ ID NO: 9. In some embodiments, an elabela analogue comprises sequence that is at least 90% identical to SEQ ID NO: 9. In some embodiments, an elabela analogue comprises sequence that is at least 96% identical to SEQ ID NO: 9. In some embodiments, an elabela analogue comprises sequence that is at least 97% identical to SEQ ID NO: 9. In some embodiments, an elabela analogue comprises sequence that is at least 98% identical to SEQ ID NO: 9. In some embodiments, an elabela analogue comprises sequence that is at least 99% identical to SEQ ID NO: 9.

In some embodiments, an elabela analogue comprises sequence that is at least 90% identical to SEQ ID NO: 10. In some embodiments, an elabela analogue comprises sequence that is at least 92% identical to SEQ ID NO: 10. In some embodiments, an elabela analogue comprises sequence that is at least 95% identical to SEQ ID NO: 10. In some embodiments, an elabela analogue comprises sequence that is at least 90% identical to SEQ ID NO: 10. In some embodiments, an elabela analogue comprises sequence that is at least 96% identical to SEQ ID NO: 10. In some embodiments, an elabela analogue comprises sequence that is at least 97% identical to SEQ ID NO: 10. In some embodiments, an elabela analogue comprises sequence that is at least 98% identical to SEQ ID NO: 10. In some embodiments, an elabela analogue Comprises sequence that is at least 99% identical to SEQ ID NO: 10.

In some embodiments, an elabela analogue comprises sequence that is at least 90% identical to SEQ ID NO: 11. In some embodiments, an elabela analogue comprises sequence that is at least 92% identical to SEQ ID NO: 11. In some embodiments, an elabela analogue comprises sequence that is at least 95% identical to SEQ ID NO: 11. In some embodiments, an elabela analogue comprises sequence that is at least 90% identical to SEQ ID NO: 11. In some embodiments, an elabela analogue comprises sequence that is at least 96% identical to SEQ ID NO: 11. In some embodiments, an elabela analogue comprises sequence that is at least 97% identical to SEQ ID NO: 11. In some embodiments, an elabela analogue comprises sequence that is at least 98% identical to SEQ ID NO: 11. In some embodiments, an elabela analogue comprises sequence that is at least 99% identical to SEQ ID NO: 11.

In some embodiments, an elabela analogue comprises sequence that is at least 90% identical to SEQ ID NO: 12. In some embodiments, an elabela analogue comprises sequence that is at least 92% identical to SEQ ID NO: 12. In some embodiments, an elabela analogue comprises sequence that is at least 95% identical to SEQ ID NO: 12. In some embodiments, an elabela analogue comprises sequence that is at least 90% identical to SEQ ID NO: 12. In some embodiments, an elabela analogue comprises sequence that is at least 96% identical to SEQ ID NO: 12. In some embodiments, an elabela analogue comprises sequence that is at least 97% identical to SEQ ID NO: 12. In some embodiments, an elabela analogue comprises sequence that is at least 98% identical to SEQ ID NO: 8. In some embodiments, an elabela analogue comprises sequence that is at least 99% identical to SEQ ID NO: 12.

In some embodiments, an elabela analogue comprises sequence that is at least 90% identical to SEQ ID NO: 13. In some embodiments, an elabela analogue comprises sequence that is at least 92% identical to SEQ ID NO: 13. In some embodiments, an elabela analogue comprises sequence that is at least 95% identical to SEQ ID NO: 13. In some embodiments, an elabela analogue comprises sequence that is at least 90% identical to SEQ ID NO: 13. In some embodiments, an elabela analogue comprises sequence that is at least 96% identical to SEQ ID NO: 13. In some embodiments, an elabela analogue comprises sequence that is at least 97% identical to SEQ ID NO: 13. In some embodiments, an elabela analogue comprises sequence that is at least 98% identical to SEQ ID NO: 13. In some embodiments, an elabela analogue comprises sequence that is at least 99% identical to SEQ ID NO: 13.

In some embodiments, an elabela analogue comprises sequence that is at least 90% identical to SEQ ID NO: 30. In some embodiments, an elabela analogue comprises sequence that is at least 92% identical to SEQ ID NO: 30. In some embodiments, an elabela analogue comprises sequence that is at least 95% identical to SEQ ID NO: 30. In some embodiments, an elabela analogue comprises sequence that is at least 90% identical to SEQ ID NO: 30. In some embodiments, an elabela analogue comprises sequence that is at least 96% identical to SEQ ID NO: 30. In some embodiments, an elabela analogue comprises sequence that is at least 97% identical to SEQ ID NO: 30. In some embodiments, an elabela analogue comprises sequence that is at least 98% identical to SEQ ID NO: 30. In some embodiments, an elabela analogue comprises sequence that is at least 99% identical to SEQ ID NO: 30.

In some embodiments, an elabela analogue comprises sequence that is at least 90% identical to SEQ ID NO: 31. In some embodiments, an elabela analogue comprises sequence that is at least 92% identical to SEQ ID NO: 31. In some embodiments, an elabela analogue comprises sequence that is at least 95% identical to SEQ ID NO: 31. In some embodiments, an elabela analogue comprises sequence that is at least 90% identical to SEQ ID NO: 31. In some embodiments, an elabela analogue comprises sequence that is at least 96% identical to SEQ ID NO: 31. In some embodiments, an elabela analogue comprises sequence that is at least 97% identical to SEQ ID NO: 31. In some embodiments, an elabela analogue comprises sequence that is at least 98% identical to SEQ ID NO: 31. In some embodiments, an elabela analogue comprises sequence that is at least 99% identical to SEQ ID NO: 31.

In some embodiments, an elabela analogue comprises sequence that is at least 90% identical to SEQ ID NO: 32. In some embodiments, an elabela analogue comprises sequence that is at least 92% identical to SEQ ID NO: 32. In some embodiments, an elabela analogue comprises sequence that is at least 95% identical to SEQ ID NO: 32. In some embodiments, an elabela analogue comprises sequence that is at least 90% identical to SEQ ID NO: 32. In some embodiments, an elabela analogue comprises sequence that is at least 96% identical to SEQ ID NO: 32. In some embodiments, an elabela analogue comprises sequence that is at least 97% identical to SEQ ID NO: 32. In some embodiments, an elabela analogue comprises sequence that is at least 98% identical to SEQ ID NO: 32. In some embodiments, an elabela analogue Comprises sequence that is at least 99% identical to SEQ ID NO: 32.

In some embodiments, an elabela analogue comprises sequence that is at least 90% identical to SEQ ID NO: 33. In some embodiments, an elabela analogue comprises sequence that is at least 92% identical to SEQ ID NO: 33. In some embodiments, an elabela analogue comprises sequence that is at least 95% identical to SEQ ID NO: 33. In some embodiments, an elabela analogue comprises sequence that is at least 90% identical to SEQ ID NO: 33. In some embodiments, an elabela analogue comprises sequence that is at least 96% identical to SEQ ID NO: 33. In some embodiments, an elabela analogue comprises sequence that is at least 97% identical to SEQ ID NO: 33. In some embodiments, an elabela analogue comprises sequence that is at least 98% identical to SEQ ID NO: 33. In some embodiments, an elabela analogue Comprises sequence that is at least 99% identical to SEQ ID NO: 33.

In some embodiments, an elabela analogue comprises sequence that is at least 90% identical to SEQ ID NO: 34. In some embodiments, an elabela analogue comprises sequence that is at least 92% identical to SEQ ID NO: 34. In some embodiments, an elabela analogue comprises sequence that is at least 95% identical to SEQ ID NO: 34. In some embodiments, an elabela analogue comprises sequence that is at least 90% identical to SEQ ID NO: 34. In some embodiments, an elabela analogue comprises sequence that is at least 96% identical to SEQ ID NO: 34. In some embodiments, an elabela analogue comprises sequence that is at least 97% identical to SEQ ID NO: 34. In some embodiments, an elabela analogue comprises sequence that is at least 98% identical to SEQ ID NO: 34. In some embodiments, an elabela analogue Comprises sequence that is at least 99% identical to SEQ ID NO: 34.

In some embodiments, an elabela analogue comprises sequence that is at least 90% identical to SEQ ID NO: 35. In some embodiments, an elabela analogue comprises sequence that is at least 92% identical to SEQ ID NO: 35. In some embodiments, an elabela analogue comprises sequence that is at least 95% identical to SEQ ID NO: 35. In some embodiments, an elabela analogue comprises sequence that is at least 90% identical to SEQ ID NO: 35. In some embodiments, an elabela analogue comprises sequence that is at least 96% identical to SEQ ID NO: 35. In some embodiments, an elabela analogue comprises sequence that is at least 97% identical to SEQ ID NO: 35. In some embodiments, an elabela analogue comprises sequence that is at least 98% identical to SEQ ID NO: 35. In some embodiments, an elabela analogue comprises sequence that is at least 99% identical to SEQ ID NO: 35.

In some embodiments, an apelin analogue comprises the amino acid sequence

(SEQ ID NO: 67)
K-L-Z1-K-H-N-Z2-Z3-Z4-Z5-R-
Z6-M-Z7-Z8-H-Z9-Z10-V-P-F-P

Z1 is R or N;
Z2 is C or S;
Z3 is A or L;
Z4 is Q, I, K, or T;
Z5 is R or E;
Z6 is C or Y;
Z7 is P or I;
Z8 is L or Y;
Z9 is S, Q, or R; and/or
Z10 is R or Y,

### Carrier Moiety

The present invention provides, among other things, an apelin receptor agonist comprising an apelin analogue or an elabela analogue fused to a carrier moiety.

### Half-Life Extension

In some embodiments, the carrier moiety extends a half-life of the apelin receptor agonist. As peptides, apelin and elabela have half-lives on the order of minutes. The short half-life is partially mediated by proteolytic degradation by a number of circulating proteases that recognize and cleave at specific sequences. In addition to proteolytic degradation, peptides are also rapidly cleared from the blood through the kidneys. In some embodiments, a carrier moiety prevents the apelin receptor agonist from renal clearance. In some embodiments, a carrier moiety protects the apelin receptor agonist from proteolysis.

### Fc Domain

In some embodiments, a carrier moiety is an Fc domain. In some embodiments, a carrier moiety is an Fc domain of IgG1. In some embodiments, a carrier moiety is an Fc domain of IgG2. In some embodiments, a carrier moiety is an Fc domain of IgG3. In some embodiments, a carrier moiety is an Fc domain of IgG4.

In some embodiments, an Fc domain has reduced or abolished effector functions. In some embodiments, an Fc domain has reduced or abolished binding to all FcγRI, FcγRIIa, FcγRIIb, FcγRIIIa, FcγRIIIb, and C1q, and maintain its ability to bind to FcRn.

In some embodiments, an Fc domain is a modified Fc domain comprising one or more mutations. The Fc variants of the present invention may be optimized for a variety of properties. An Fc variant that is engineered or predicted to display one or more optimized properties is herein referred to as an “optimized Fc variant”. Properties that may be optimized include but are not limited to enhanced or reduced affinity for an FcγR. In some embodiments, the Fc variants of the present invention are optimized to have reduced or ablated affinity for a human FcγR, including but not limited to FcγRI, FcγRIIa, FcγRIIb, FcγRIIc, FcγRIIIa, and FcγRIIIb. These embodiments are anticipated to provide Fc polypeptides with enhanced therapeutic properties in humans, for example reduced effector function and reduced toxicity. In other embodiments, Fc variants of the present invention provide enhanced affinity for one or more FcγRs, yet reduced affinity for one or more other FcγRs. F or example, an Fc variant of the present invention may have enhanced binding to FcγRIIIa, yet reduced binding to FcγRIIb. Alternately, an Fc variant of the present invention may have enhanced binding to FcγRIIa and FcγRI, yet reduced binding to FcγRIIb. In yet another embodiment, an Fc variant of the present invention may have enhanced affinity for FcγRIIb, yet reduced affinity to one or more activating FcγRs.

In some embodiments, an Fc variant has reduced or ablated affinity for FcγRI. In some embodiments, an Fc variant has reduced or ablated affinity for FcγRIIa. In some embodiments, an Fc variant has reduced or ablated affinity for FcγRIIb. In some embodiments, an Fc variant has reduced or ablated affinity for FcγRIIc. In some embodiments, an Fc variant has reduced or ablated affinity for FcγRIIIa. In some embodiments, an Fc variant has reduced or ablated affinity for FcγRIIIb. In some embodiments, an Fc variant has reduced or ablated affinity for C1q. In some embodiments, an Fc variant has enhanced affinity for FcRn. In some embodiments, an Fc variant maintains affinity for FcRn. In some embodiments, an Fc variant has reduced or ablated affinity for FcγRI, FcγRIIa, FcγRIIb, FcγRIIIa, FcγRIIIb, and C1q. In some embodiments, an Fc variant has reduced or ablated affinity for FcγRI, FcγRIIa, FcγRIIb, FcγRIIIa, FcγRIIIb, and C1q, and retains binding to FcRn.

In some embodiments, an Fc domain is a monovalent Fc domain. In some embodiments, an Fc domain is a human IgG1 monovalent Fc domain. In some embodiments, an Fc domain is a human IgG2 monovalent Fc domain. In some embodiments, an Fc domain is a human IgG3 monovalent Fc domain. In some embodiments, an Fc domain is a human IgG4 monovalent Fc domain. In some embodiments, an Fc domain is a modified human IgG1 monovalent Fc domain. In some embodiments, an Fc domain is a modified human IgG2 monovalent Fc domain. In some embodiments, an Fc domain is a modified human IgG3 monovalent Fc domain. In some embodiments, an Fc domain is a modified human IgG4 monovalent Fc domain.

In some embodiments, an Fc domain is a bivalent Fc domain. In some embodiments, an Fc domain is a human IgG1 bivalent Fc domain. In some embodiments, an Fc domain is a human IgG2 bivalent Fc domain. In some embodiments, an Fc domain is a human IgG3 bivalent Fc domain. In some embodiments, an Fc domain is a human IgG4 bivalent Fc domain. In some embodiments, an Fc domain is a modified human IgG1 bivalent Fc domain. In some embodiments, an Fc domain is a modified human IgG2 bivalent Fc domain. In some embodiments, an Fc domain is a modified human IgG3 bivalent Fc domain. In some embodiments, an Fc domain is a modified human IgG4 bivalent Fc domain. In some embodiments, the modified human IgG4 bivalent Fc domain comprises S228P substitution.

In some embodiments, a carrier moiety comprises SEQ ID NO: 14. In some embodiments, an Fc domain of IgG1 comprises SEQ ID NO: 14. In some embodiments, a human IgG1 monovalent Fc domain comprises SEQ ID NO: 14.

(SEQ ID NO: 14)
DKTHTSPPSPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVNLTCLVKGFYPSDIAVEWESNGQPENNYKTTPPVLDS
DGSFFLNSTLTVDKSRWQQGNVFSCSVMHEALHNHYTQKSLSLSP
GK

In some embodiments, a carrier moiety comprises amino acid sequence that is at least 80% identical to SEQ ID NO: 14. In some embodiments, a carrier moiety comprises amino acid sequence that is at least 85% identical to SEQ ID NO: 14. In some embodiments, a carrier moiety comprises amino acid sequence that is at least 88% identical to SEQ ID NO: 14. In some embodiments, a carrier moiety comprises amino acid sequence that is at least 90% identical to SEQ ID NO: 14. In some embodiments, a carrier moiety comprises amino acid sequence that is at least 92% identical to SEQ ID NO: 14. In some embodiments, a carrier moiety comprises amino acid sequence that is at least 93% identical to SEQ ID NO: 14. In some embodiments, a carrier moiety comprises amino acid sequence that is at least 94% identical to SEQ ID NO: 14. In some embodiments, a carrier moiety comprises amino acid sequence that is at least 95% identical to SEQ ID NO: 14. In some embodiments, a carrier moiety comprises amino acid sequence that is at least 96% identical to SEQ ID NO: 14. In some embodiments, a carrier moiety comprises amino acid sequence that is at least 97% identical to SEQ ID NO: 14. In some embodiments, a carrier moiety comprises amino acid sequence that is at least 98% identical to SEQ ID NO: 14. In some embodiments, a carrier moiety comprises amino acid sequence that is at least 99% identical to SEQ ID NO: 14.

In some embodiments, a carrier moiety comprises SEQ ID NO: 15. In some embodiments, an Fc domain of IgG1 comprises SEQ ID NO: 15. In some embodiments, a human IgG1 monovalent Fc domain comprises SEQ ID NO: 15.

(SEQ ID NO: 15)
MRAWIFFLLCLAGRALADKTHTSPPSPAPEAAGGPSVFLFPPKPK
DTLMISRTPEVTCVVVDVSHEDPEVKFNWYVDGVEVHNAKTKPRE
EQYNSTYRVVSVLTVLHQDWLNGKEYKCKVSNKALPAPIEKTISK
AKGQPREPQVYTLPPSREEMTKNQVNLTCLVKGFYPSDIAVEWES
NGQPENNYKTTPPVLDSDGSFFLNSTLTVDKSRWQQGNVFSCSVM
HEALHNHYTQKSLSLSPGK

In some embodiments, a carrier moiety comprises amino acid sequence that is at least 80% identical to SEQ ID NO: 15. In some embodiments, a carrier moiety comprises amino acid sequence that is at least 85% identical to SEQ ID NO: 15. In some embodiments, a carrier moiety comprises amino acid sequence that is at least 88% identical to SEQ ID NO: 15. In some embodiments, a carrier moiety comprises amino acid sequence that is at least 90% identical to SEQ ID NO: 15. In some embodiments, a carrier moiety comprises amino acid sequence that is at least 92% identical to SEQ ID NO: 15. In some embodiments, a carrier moiety comprises amino acid sequence that is at least 93% identical to SEQ ID NO: 15. In some embodiments, a carrier moiety comprises amino acid sequence that is at least 94% identical to SEQ ID NO: 15. In some embodiments, a carrier moiety comprises amino acid sequence that is at least 95% identical to SEQ ID NO: 15. In some embodiments, a carrier moiety comprises amino acid sequence that is at least 96% identical to SEQ ID NO: 15. In some embodiments, a carrier moiety comprises amino acid sequence that is at least 97% identical to SEQ ID NO: 15. In some embodiments, a carrier moiety comprises amino acid sequence that is at least 98% identical to SEQ ID NO: 15. In some embodiments, a carrier moiety comprises amino acid sequence that is at least 99% identical to SEQ ID NO: 15.

### Other Carrier Moieties

In some embodiments, a carrier moiety comprises a serum protein binding moiety. In some embodiments, a carrier moiety is a human serum albumin (HSA). In some embodiments, a carrier moiety comprises a protein or a peptide that binds to HSA. In some embodiments, a carrier moiety comprises an anti-HSA antibody or a fragment thereof. In some embodiments, a carrier moiety comprises an anti-HSA Fab. In some embodiments, a carrier moiety comprises an anti-HSA nanobody (VHH). In some embodiments, a carrier moiety comprises an anti-HAS scFv. In some embodiments, a carrier moiety comprises an anti-HSA V-NAR. In some embodiments, a carrier moiety comprises an anti-HSA VHH of ALB-23. ALB-23 is described in WO2012175400A1, the contents of which are incorporated by reference.

In some embodiments, a carrier moiety comprises one or more lipids. Lipids can be of or comprise various lengths, chemistries, linkers, and conjugates that are apparent to one skilled in the art. In some embodiments, a carrier moiety comprises a saturated fatty acid. In some embodiments, the saturated fatty acid is linked to the peptide through its carboxylate group. In some embodiments, the saturated fatty acid comprises a C₆₋₁₈ saturated fatty acid (e.g., a C₆, C₁₂, C₁₆, or C₁₈ saturated fatty acid). In some embodiments, a carrier moiety comprises a saturated fatty diacid. In some embodiments, the saturated fatty diacid is linked to the peptide through a carboxylate group its free end. In some embodiments, the saturated fatty diacid comprises a C₆₋₁₈ saturated fatty diacid (e.g., a C₆, C₁₂, C₁₆, or C₁₈ saturated fatty diacid).

In some embodiments, a carrier moiety comprises one or more polyethylene glycols (PEGs). PEGs can be of or comprise various lengths, various molecular weights and linkers that are apparent to one skilled in the art. In some embodiments, the PEG is monodisperse. In some embodiments, the monodisperse PEG has 12-48 ethylene glycol repeats (e.g., 12-24 repeats, 12-36 repeats, 24-48 repeats, 24-36 repeats, or 36-48 repeats). In some embodiments, the PEG is polydisperse. In some embodiments, the polydisperse PEG has a molecular weight of 500 to 50,000 daltons (e.g., 500-1,000 Da, 500-5,000 Da, 500-20,000 Da, 500-40,000 Da, 1,000-5,000 Da, 1,000-20,000 Da, 1,000-40,000 Da, 1,000-50,000 Da, 5,000-10,000 Da, 5,000-20,000 Da, 5,000-40,000 Da, 5,000-50,000 Da, 10,000-20,000 Da, 10,000-40,000 Da, 10,000-50,000 Da, 20,000-40,000 Da, or 20,000-50,000 Da). In some embodiments, the polydisperse PEG has a molecular weight of 0.5 kDa, 1 kDa, 5 kDa, 10 kDa, 15 kDa, 20 kDa, 25 kDa, 30 kDa, 35 kDa, 40 kDa, 45 kDa, or 50 kDa. In some embodiments, a carrier moiety comprises a polydisperse PEG with a molecular weight of 1 kDa. In some embodiments, a carrier moiety comprises a polydisperse PEG with a molecular weight of 5 kDa. In some embodiments, a carrier moiety comprises a polydisperse PEG with a molecular weight of 10 kDa. In some embodiments, a carrier moiety comprises a polydisperse PEG with a molecular weight of 15 kDa. In some embodiments, a carrier moiety comprises a polydisperse PEG with a molecular weight of 20 kDa. In some embodiments, a carrier moiety comprises a polydisperse PEG with a molecular weight of 25 kDa. In some embodiments, a carrier moiety comprises a polydisperse PEG with a molecular weight of 30 kDa. In some embodiments, a carrier moiety comprises a polydisperse PEG with a molecular weight of 35 kDa. In some embodiments, a carrier moiety comprises a polydisperse PEG with a molecular weight of 40 kDa. In some embodiments, a carrier moiety comprises a polydisperse PEG with a molecular weight of 50 kDa.

In some embodiments, a carrier moiety comprises a half-life extending polypeptide. Half-life extending polypeptides can be of or comprise various lengths, chemistries, linkers, and conjugates that are apparent to one skilled in the art. In some embodiment, the half-life extending polypeptide comprises repeated units of Pro-Ala-Ser (PAS). In some embodiments, the half-life extending polypeptide comprises 50-500 repeated PAS units (e.g., 50-100 PAS units, 50-200 PAS units, 100-200 PAS units, 100-500 PAS units, or 200-500 PAS units).

In some embodiments, the half-life extending polypeptide comprises an XTEN™ polypeptide. XTEN is described in US20150037359A1, the contents of which are incorporated by reference.

In some embodiments, an apelin analogue or an elabela analogue is not fused to a carrier moiety. In some embodiments, an apelin analogue or an elabela analogue exists as a free peptide.

### Exemplary Apelin Receptor Agonists

The present invention provides, among other things, an apelin receptor agonist comprising an apelin analogue or an elabela analogue fused to a carrier moiety via a linker. The present invention provides, among other things, an apelin receptor agonist comprising an apelin analogue or an elabela analogue, wherein the N-terminus of the apelin analogue or the elabela analogue is fused to a carrier moiety via a linker. In some embodiments, the N-terminus of the apelin analogue of the elabela analogue is fused to the C-terminus of the carrier moiety. In some embodiments, the N-terminus of the apelin analogue of the elabela analogue is fused to the C-terminus of an Fc domain. In some embodiments, the N-terminus of the apelin analogue of the elabela analogue is fused to the C-terminus of a monovalent Fc domain. In some embodiments, the N-terminus of the apelin analogue of the elabela analogue is fused to the C-terminus of a monovalent IgG1 Fc domain.

In some embodiments, a linker comprises glycine and serine residues. In some embodiments, a linker consists of glycine and serine residues. In some embodiments, a linker comprises amino acid sequence of SEQ ID NO: 16 (GGGGSGGGGSGGGGS).

In some embodiments, an apelin receptor agonist comprises a sequence shown in Table 3.

TABLE 3
Exemplary Apelin Receptor Agonists

| Apelin | | | SEQ |
|---|---|---|---|
| Receptor | | APJ | ID |
| Agonist | Sequence | ligand | NO |
| APL-A | DKTHTSPPSPAPEAAGGPSVFLFPPKPKDTLMISRT | Apelin | 17 |
| | PEVTCVVVDVSHEDPEVKFNWYVDGVEVHNAKTKPR | | |
| | EEQYNSTYRVVSVLTVLHQDWLNGKEYKCKVSNKA | | |
| | LPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSQRPRLSH | | |
| | KGPMPF | | |
| APL-B | DKTHTSPPSPAPEAAGGPSVFLFPPKPKDTLMISRT | Apelin | 18 |
| | PEVTCVVVDVSHEDPEVKFNWYVDGVEVHNAKTKPR | | |
| | EEQYNSTYRVVSVLTVLHQDWLNGKEYKCKVSNKA | | |
| | LPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSQRPRLSH | | |
| | KGPMVF | | |
| APL-C | DKTHTSPPSPAPEAAGGPSVFLFPPKPKDTLMISRT | Apelin | 19 |
| | PEVTCVVVDVSHEDPEVKFNWYVDGVEVHNAKTKPR | | |
| | EEQYNSTYRVVSVLTVLHQDWLNGKEYKCKVSNKA | | |
| | LPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSQRPRLSH | | |
| | WGPMPF | | |
| APL-D | DKTHTSPPSPAPEAAGGPSVFLFPPKPKDTLMISRT | Apelin | 20 |
| | PEVTCVVVDVSHEDPEVKFNWYVDGVEVHNAKTKPR | | |
| | EEQYNSTYRVVSVLTVLHQDWLNGKEYKCKVSNKA | | |
| | LPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSERPRLSHK | | |
| | GPMPF | | |
| APL-E | DKTHTSPPSPAPEAAGGPSVFLFPPKPKDTLMISRT | Apelin | 21 |
| | PEVTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSQRPRLSH | | |
| | KGPHPF | | |
| APL-F | DKTHTSPPSPAPEAAGGPSVFLFPPKPKDTLMISRT | Apelin | 22 |
| | PEVTCVVVDVSHEDPEVKFNWYVDGVEVHNAKTKPR | | |
| | EEQYNSTYRVVSVLTVLHQDWLNGKEYKCKVSNKA | | |
| | LPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSQRPRLSH | | |
| | KGPAPY | | |
| APL-G | DKTHTSPPSPAPEAAGGPSVFLFPPKPKDTLMISRT | Apelin | 23 |
| | PEVTCVVVDVSHEDPEVKFNWYVDGVEVHNAKTKPR | | |
| | EEQYNSTYRVVSVLTVLHQDWLNGKEYKCKVSNKA | | |
| | LPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSQRPRLSH | | |
| | KGPMAF | | |
| APL-H | MRAWIFFLLCLAGRALADKTHTSPPSPAPEAAGGPS | Apelin | 56 |
| | VFLFPPKPKDTLMISRTPEVTCVVVDVSHEDPEVKE | | |
| | WNYVDGVEVHNAKTKPREEQYNSTYRVVSVLTVLH | | |
| | QDWLNGKEYKCKVSNKALPAPIEKTISKAKGQPREP | | |
| | QVYTLPPSREEMTKNQVNLTCLVKGFYPSDIAVEWE | | |
| | SNGQPENNYKTTPPVLDSDGSFFLNSTLTVDKSRWQ | | |
| | QGNVFSCSVMHEALHNHYTQKSLSLSPGKGGGGSG | | |
| | GGGSGGGGSQRPRLSHKGPMHW | | |
| APL-I | MRAWIFFLLCLAGRALADKTHTSPPSPAPEAAGGPS | Apelin | 57 |
| | VFLFPPKPKDTLMISRTPEVTCVVVDVSHEDPEVKF | | |
| | NWYVDGVEVHNAKTKPREEQYNSTYRVVSVLTVLH | | |
| | QDWLNGKEYKCKVSNKALPAPIEKTISKAKGQPREP | | |
| | QVYTLPPSREEMTKNQVNLTCLVKGFYPSDIAVEWE | | |
| | SNGQPENNYKTTPPVLDSDGSFFLNSTLTVDKSRWQ | | |
| | QGNVFSCSVMHEALHNHYTQKSLSLSPGKGGGGSG | | |
| | GGGSGGGGSQRPRLSHKGPAAW | | |
| APL-J | MRAWIFFLLCLAGRALADKTHTSPPSPAPEAAGGPS | Apelin | 58 |
| | VFLFPPKPKDTLMISRTPEVTCVVVDVSHEDPEVKF | | |
| | NWYVDGVEVHNAKTKPREEQYNSTYRVVSVLTVLH | | |
| | QDWLNGKEYKCKVSNKALPAPIEKTISKAKGQPREP | | |
| | QVYTLPPSREEMTKNQVNLTCLVKGFYPSDIAVEWE | | |
| | SNGQPENNYKTTPPVLDSDGSFFLNSTLTVDKSRWQ | | |
| | QGNVFSCSVMHEALHNHYTQKSLSLSPGKGGGGSG | | |
| | GGGSGGGGSQRPRLSHKGPAAY | | |
| APL-K | MRAWIFFLLCLAGRALADKTHTSPPSPAPEAAGGPS | Apelin | 59 |
| | VFLFPPKPKDTLMISRTPEVTCVVVDVSHEDPEVKE | | |
| | NWYVDGVEVHNAKTKPREEQYNSTYRVVSVLTVLH | | |
| | QDWLNGKEYKCKVSNKALPAPIEKTISKAKGQPREP | | |
| | QVYTLPPSREEMTKNQVNLTCLVKGFYPSDIAVEWE | | |
| | SNGQPENNYKTTPPVLDSDGSFFLNSTLTVDKSRWQ | | |
| | QGNVFSCSVMHEALHNHYTQKSLSLSPGKGGGGSG | | |
| | GGGSGGGGSQRPRLQHKNPMAF | | |
| APL-L | MRAWIFFLLCLAGRALADKTHTSPPSPAPEAAGGPS | Apelin | 60 |
| | VFLFPPKPKDTLMISRTPEVTCVVVDVSHEDPEVKE | | |
| | NWYVDGVEVHNAKTKPREEQYNSTYRVVSVLTVLH | | |
| | QDWLNGKEYKCKVSNKALPAPIEKTISKAKGQPREP | | |
| | QVYTLPPSREEMTKNQVNLTCLVKGFYPSDIAVEWE | | |
| | SNGQPENNYKTTPPVLDSDGSFFLNSTLTVDKSRWQ | | |
| | QGNVFSCSVMHEALHNHYTQKSLSLSPGKGGGGSG | | |
| | GGGSGGGGSQRPRLQHKNPMAW | | |
| APL-M | MRAWIFFLLCLAGRALADKTHTSPPSPAPEAAGGPS | Apelin | 61 |
| | VFLFPPKPKDTLMISRTPEVTCVVVDVSHEDPEVKE | | |
| | NWYVDGVEVHNAKTKPREEQYNSTYRVVSVLTVLH | | |
| | QDWLNGKEYKCKVSNKALPAPIEKTISKAKGQPREP | | |
| | QVYTLPPSREEMTKNQVNLTCLVKGFYPSDIAVEWE | | |
| | SNGQPENNYKTTPPVLDSDGSFFLNSTLTVDKSRWQ | | |
| | QGNVFSCSVMHEALHNHYTQKSLSLSPGKGGGGSG | | |
| | GGGSGGGGSQRPRLSHWGPMAW | | |
| ELA-A | DKTHTSPPSPAPEAAGGPSVFLFPPKPKDTLMISRTPE | Elabela | 24 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQRRCMPLHSRVPFP | | |
| ELA-B | DKTHTSPPSPAPEAAGGPSVFLFPPKPKDTLMISRTPE | Elabela | 25 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLNKHNC | | |
| | LQRRCMPLHSRVPFP | | |
| ELA-C | DKTHTSPPSPAPEAAGGPSVFLFPPKPKDTLMISRTPE | Elabela | 26 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQRRCMILHSRVPFP | | |
| ELA-D | DKTHTSPPSPAPEAAGGPSVFLFPPKPKDTLMISRTPE | Elabela | 27 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQRRCMPLHQRVPFP | | |
| ELA-E | DKTHTSPPSPAPEAAGGPSVFLFPPKPKDTLMISRTPE | Elabela | 28 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQRRCMPLHRRVPFP | | |
| ELA-F | DKTHTSPPSPAPEAAGGPSVFLFPPKPKDTLMISRTPE | Elabela | 29 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LKRRCMPLHSRVPFP | | |
| ELA-G | DKTHTSPPSPAPEAAGGPSVFLFPPKPKDTLMISRTPE | Elabela | 36 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQRRCMPLHSYVPFP | | |
| ELA-H | DKTHTSPPSPAPEAAGGPSVFLFPPKPKDTLMISRTPE | Elabela | 37 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQRRCMPYHSRVPFP | | |
| ELA-I | DKTHTSPPSPAPEAAGGPSVFLFPPKPKDTLMISRTPE | Elabela | 38 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNS | | |
| | LQRRYMPLHSRVPFP | | |
| ELA-J | DKTHTSPPSPAPEAAGGPSVFLFPPKPKDTLMISRTPE | Elabela | 39 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LQERCMPLHSRVPFP | | |
| ELA-K | DKTHTSPPSPAPEAAGGPSVFLFPPKPKDTLMISRTPE | Elabela | 40 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | LIRRCMPLHSRVPFP | | |
| ELA-L | DKTHTSPPSPAPEAAGGPSVFLFPPKPKDTLMISRTPE | Elabela | 41 |
| | VTCVVVDVSHEDPEVKFNWYVDGVEVHNAKTKPR | | |
| | EEQYNSTYRVVSVLTVLHQDWLNGKEYKCKVSNK | | |
| | ALPAPIEKTISKAKGQPREPQVYTLPPSREEMTKNQV | | |
| | NLTCLVKGFYPSDIAVEWESNGQPENNYKTTPPVLD | | |
| | SDGSFFLNSTLTVDKSRWQQGNVFSCSVMHEALHN | | |
| | HYTQKSLSLSPGKGGGGSGGGGSGGGGSKLRKHNC | | |
| | ATRRCMPLHSRVPFP | | |

In some embodiments, an apelin analogue comprises sequence that is at least 80% identical to SEQ ID NO: 17. In some embodiments, an apelin analogue comprises sequence that is at least 82% identical to SEQ ID NO: 17. In some embodiments, an apelin analogue comprises sequence that is at least 84% identical to SEQ ID NO: 17. In some embodiments, an apelin analogue comprises sequence that is at least 86% identical to SEQ ID NO: 17. In some embodiments, an apelin analogue comprises sequence that is at least 88% identical to SEQ ID NO: 17. In some embodiments, an apelin analogue comprises sequence that is at least 90% identical to SEQ ID NO: 17. In some embodiments, an apelin analogue comprises sequence that is at least 92% identical to SEQ ID NO: 17. In some embodiments, an apelin analogue comprises sequence that is at least 95% identical to SEQ ID NO: 17. In some embodiments, an apelin analogue comprises sequence that is at least 90% identical to SEQ ID NO: 17. In some embodiments, an apelin analogue comprises sequence that is at least 96% identical to SEQ ID NO: 17. In some embodiments, an apelin analogue comprises sequence that is at least 97% identical to SEQ ID NO: 17. In some embodiments, an apelin analogue comprises sequence that is at least 98% identical to SEQ ID NO: 17. In some embodiments, an apelin analogue comprises sequence that is at least 99% identical to SEQ ID NO: 17.

In some embodiments, an apelin analogue comprises sequence that is at least 80% identical to SEQ ID NO: 18. In some embodiments, an apelin analogue comprises sequence that is at least 82% identical to SEQ ID NO: 18. In some embodiments, an apelin analogue comprises sequence that is at least 84% identical to SEQ ID NO: 18. In some embodiments, an apelin analogue comprises sequence that is at least 86% identical to SEQ ID NO: 18. In some embodiments, an apelin analogue comprises sequence that is at least 88% identical to SEQ ID NO: 18. In some embodiments, an apelin analogue comprises sequence that is at least 90% identical to SEQ ID NO: 18. In some embodiments, an apelin analogue comprises sequence that is at least 92% identical to SEQ ID NO: 18. In some embodiments, an apelin analogue comprises sequence that is at least 95% identical to SEQ ID NO: 18. In some embodiments, an apelin analogue comprises sequence that is at least 90% identical to SEQ ID NO: 18. In some embodiments, an apelin analogue comprises sequence that is at least 96% identical to SEQ ID NO: 18. In some embodiments, an apelin analogue comprises sequence that is at least 97% identical to SEQ ID NO: 18. In some embodiments, an apelin analogue comprises sequence that is at least 98% identical to SEQ ID NO: 18. In some embodiments, an apelin analogue comprises sequence that is at least 99% identical to SEQ ID NO: 18.

In some embodiments, an apelin analogue comprises sequence that is at least 80% identical to SEQ ID NO: 19. In some embodiments, an apelin analogue comprises sequence that is at least 82% identical to SEQ ID NO: 19. In some embodiments, an apelin analogue comprises sequence that is at least 84% identical to SEQ ID NO: 19. In some embodiments, an apelin analogue comprises sequence that is at least 86% identical to SEQ ID NO: 19. In some embodiments, an apelin analogue comprises sequence that is at least 88% identical to SEQ ID NO: 19. In some embodiments, an apelin analogue comprises sequence that is at least 90% identical to SEQ ID NO: 19. In some embodiments, an apelin analogue comprises sequence that is at least 92% identical to SEQ ID NO: 19. In some embodiments, an apelin analogue comprises sequence that is at least 95% identical to SEQ ID NO: 19. In some embodiments, an apelin analogue comprises sequence that is at least 90% identical to SEQ ID NO: 19. In some embodiments, an apelin analogue comprises sequence that is at least 96% identical to SEQ ID NO: 19. In some embodiments, an apelin analogue comprises sequence that is at least 97% identical to SEQ ID NO: 19. In some embodiments, an apelin analogue comprises sequence that is at least 98% identical to SEQ ID NO: 19. In some embodiments, an apelin analogue comprises sequence that is at least 99% identical to SEQ ID NO: 19.

In some embodiments, an apelin analogue comprises sequence that is at least 80% identical to SEQ ID NO: 20. In some embodiments, an apelin analogue comprises sequence that is at least 82% identical to SEQ ID NO: 20. In some embodiments, an apelin analogue comprises sequence that is at least 84% identical to SEQ ID NO: 20. In some embodiments, an apelin analogue comprises sequence that is at least 86% identical to SEQ ID NO: 20. In some embodiments, an apelin analogue comprises sequence that is at least 88% identical to SEQ ID NO: 20. In some embodiments, an apelin analogue comprises sequence that is at least 90% identical to SEQ ID NO: 20. In some embodiments, an apelin analogue comprises sequence that is at least 92% identical to SEQ ID NO: 20. In some embodiments, an apelin analogue comprises sequence that is at least 95% identical to SEQ ID NO: 20. In some embodiments, an apelin analogue comprises sequence that is at least 90% identical to SEQ ID NO: 20. In some embodiments, an apelin analogue comprises sequence that is at least 96% identical to SEQ ID NO: 20. In some embodiments, an apelin analogue comprises sequence that is at least 97% identical to SEQ ID NO: 20. In some embodiments, an apelin analogue comprises sequence that is at least 98% identical to SEQ ID NO: 20. In some embodiments, an apelin analogue comprises sequence that is at least 99% identical to SEQ ID NO: 20.

In some embodiments, an apelin analogue comprises sequence that is at least 80% identical to SEQ ID NO: 21. In some embodiments, an apelin analogue comprises sequence that is at least 82% identical to SEQ ID NO: 21. In some embodiments, an apelin analogue comprises sequence that is at least 84% identical to SEQ ID NO: 21. In some embodiments, an apelin analogue comprises sequence that is at least 86% identical to SEQ ID NO: 21. In some embodiments, an apelin analogue comprises sequence that is at least 88% identical to SEQ ID NO: 21. In some embodiments, an apelin analogue comprises sequence that is at least 90% identical to SEQ ID NO: 21. In some embodiments, an apelin analogue comprises sequence that is at least 92% identical to SEQ ID NO: 21. In some embodiments, an apelin analogue comprises sequence that is at least 95% identical to SEQ ID NO: 21. In some embodiments, an apelin analogue comprises sequence that is at least 90% identical to SEQ ID NO: 21. In some embodiments, an apelin analogue comprises sequence that is at least 96% identical to SEQ ID NO: 21. In some embodiments, an apelin analogue comprises sequence that is at least 97% identical to SEQ ID NO: 21. In some embodiments, an apelin analogue comprises sequence that is at least 98% identical to SEQ ID NO: 21. In some embodiments, an apelin analogue comprises sequence that is at least 99% identical to SEQ ID NO: 21.

In some embodiments, an apelin analogue comprises sequence that is at least 80% identical to SEQ ID NO: 22. In some embodiments, an apelin analogue comprises sequence that is at least 82% identical to SEQ ID NO: 22. In some embodiments, an apelin analogue comprises sequence that is at least 84% identical to SEQ ID NO: 22. In some embodiments, an apelin analogue comprises sequence that is at least 86% identical to SEQ ID NO: 22. In some embodiments, an apelin analogue comprises sequence that is at least 88% identical to SEQ ID NO: 22. In some embodiments, an apelin analogue comprises sequence that is at least 90% identical to SEQ ID NO: 22. In some embodiments, an apelin analogue comprises sequence that is at least 92% identical to SEQ ID NO: 22. In some embodiments, an apelin analogue comprises sequence that is at least 95% identical to SEQ ID NO: 22. In some embodiments, an apelin analogue comprises sequence that is at least 90% identical to SEQ ID NO: 22. In some embodiments, an apelin analogue comprises sequence that is at least 96% identical to SEQ ID NO: 22. In some embodiments, an apelin analogue comprises sequence that is at least 97% identical to SEQ ID NO: 22. In some embodiments, an apelin analogue comprises sequence that is at least 98% identical to SEQ ID NO: 22. In some embodiments, an apelin analogue comprises sequence that is at least 99% identical to SEQ ID NO: 22.

In some embodiments, an apelin analogue comprises sequence that is at least 80% identical to SEQ ID NO: 23. In some embodiments, an apelin analogue comprises sequence that is at least 82% identical to SEQ ID NO: 23. In some embodiments, an apelin analogue comprises sequence that is at least 84% identical to SEQ ID NO: 23. In some embodiments, an apelin analogue comprises sequence that is at least 86% identical to SEQ ID NO: 23. In some embodiments, an apelin analogue comprises sequence that is at least 88% identical to SEQ ID NO: 23. In some embodiments, an apelin analogue comprises sequence that is at least 90% identical to SEQ ID NO: 23. In some embodiments, an apelin analogue comprises sequence that is at least 92% identical to SEQ ID NO: 23. In some embodiments, an apelin analogue comprises sequence that is at least 95% identical to SEQ ID NO: 23. In some embodiments, an apelin analogue comprises sequence that is at least 90% identical to SEQ ID NO: 23. In some embodiments, an apelin analogue comprises sequence that is at least 96% identical to SEQ ID NO: 23. In some embodiments, an apelin analogue comprises sequence that is at least 97% identical to SEQ ID NO: 23. In some embodiments, an apelin analogue comprises sequence that is at least 98% identical to SEQ ID NO: 23. In some embodiments, an apelin analogue comprises sequence that is at least 99% identical to SEQ ID NO: 23.

In some embodiments, an apelin analogue comprises sequence that is at least 80% identical to SEQ ID NO: 24. In some embodiments, an apelin analogue comprises sequence that is at least 82% identical to SEQ ID NO: 24. In some embodiments, an apelin analogue comprises sequence that is at least 84% identical to SEQ ID NO: 24. In some embodiments, an apelin analogue comprises sequence that is at least 86% identical to SEQ ID NO: 24. In some embodiments, an apelin analogue comprises sequence that is at least 88% identical to SEQ ID NO: 24. In some embodiments, an apelin analogue comprises sequence that is at least 90% identical to SEQ ID NO: 24. In some embodiments, an apelin analogue comprises sequence that is at least 92% identical to SEQ ID NO: 24. In some embodiments, an apelin analogue comprises sequence that is at least 95% identical to SEQ ID NO: 24. In some embodiments, an apelin analogue comprises sequence that is at least 90% identical to SEQ ID NO: 24. In some embodiments, an apelin analogue comprises sequence that is at least 96% identical to SEQ ID NO: 24. In some embodiments, an apelin analogue comprises sequence that is at least 97% identical to SEQ ID NO: 24. In some embodiments, an apelin analogue comprises sequence that is at least 98% identical to SEQ ID NO: 24. In some embodiments, an apelin analogue comprises sequence that is at least 99% identical to SEQ ID NO: 24.

In some embodiments, an apelin analogue comprises sequence that is at least 80% identical to SEQ ID NO: 25. In some embodiments, an apelin analogue comprises sequence that is at least 82% identical to SEQ ID NO: 25. In some embodiments, an apelin analogue comprises sequence that is at least 84% identical to SEQ ID NO: 25. In some embodiments, an apelin analogue comprises sequence that is at least 86% identical to SEQ ID NO: 25. In some embodiments, an apelin analogue comprises sequence that is at least 88% identical to SEQ ID NO: 25. In some embodiments, an apelin analogue comprises sequence that is at least 90% identical to SEQ ID NO: 25. In some embodiments, an apelin analogue comprises sequence that is at least 92% identical to SEQ ID NO: 25. In some embodiments, an apelin analogue comprises sequence that is at least 95% identical to SEQ ID NO: 25. In some embodiments, an apelin analogue comprises sequence that is at least 90% identical to SEQ ID NO: 25. In some embodiments, an apelin analogue comprises sequence that is at least 96% identical to SEQ ID NO: 25. In some embodiments, an apelin analogue comprises sequence that is at least 97% identical to SEQ ID NO: 25. In some embodiments, an apelin analogue comprises sequence that is at least 98% identical to SEQ ID NO: 25. In some embodiments, an apelin analogue comprises sequence that is at least 99% identical to SEQ ID NO: 25.

In some embodiments, an apelin analogue comprises sequence that is at least 80% identical to SEQ ID NO: 26. In some embodiments, an apelin analogue comprises sequence that is at least 82% identical to SEQ ID NO: 26. In some embodiments, an apelin analogue comprises sequence that is at least 84% identical to SEQ ID NO: 26. In some embodiments, an apelin analogue comprises sequence that is at least 86% identical to SEQ ID NO: 26. In some embodiments, an apelin analogue comprises sequence that is at least 88% identical to SEQ ID NO: 26. In some embodiments, an apelin analogue comprises sequence that is at least 90% identical to SEQ ID NO: 26. In some embodiments, an apelin analogue comprises sequence that is at least 92% identical to SEQ ID NO: 26. In some embodiments, an apelin analogue comprises sequence that is at least 95% identical to SEQ ID NO: 26. In some embodiments, an apelin analogue comprises sequence that is at least 90% identical to SEQ ID NO: 26. In some embodiments, an apelin analogue comprises sequence that is at least 96% identical to SEQ ID NO: 26. In some embodiments, an apelin analogue comprises sequence that is at least 97% identical to SEQ ID NO: 26. In some embodiments, an apelin analogue comprises sequence that is at least 98% identical to SEQ ID NO: 26. In some embodiments, an apelin analogue comprises sequence that is at least 99% identical to SEQ ID NO: 26.

In some embodiments, an apelin analogue comprises sequence that is at least 80% identical to SEQ ID NO: 27. In some embodiments, an apelin analogue comprises sequence that is at least 82% identical to SEQ ID NO: 27. In some embodiments, an apelin analogue comprises sequence that is at least 84% identical to SEQ ID NO: 27. In some embodiments, an apelin analogue comprises sequence that is at least 86% identical to SEQ ID NO: 27. In some embodiments, an apelin analogue comprises sequence that is at least 88% identical to SEQ ID NO: 27. In some embodiments, an apelin analogue comprises sequence that is at least 90% identical to SEQ ID NO: 27. In some embodiments, an apelin analogue comprises sequence that is at least 92% identical to SEQ ID NO: 27. In some embodiments, an apelin analogue comprises sequence that is at least 95% identical to SEQ ID NO: 27. In some embodiments, an apelin analogue comprises sequence that is at least 90% identical to SEQ ID NO: 27. In some embodiments, an apelin analogue comprises sequence that is at least 96% identical to SEQ ID NO: 27. In some embodiments, an apelin analogue comprises sequence that is at least 97% identical to SEQ ID NO: 27. In some embodiments, an apelin analogue comprises sequence that is at least 98% identical to SEQ ID NO: 27. In some embodiments, an apelin analogue comprises sequence that is at least 99% identical to SEQ ID NO: 27.

In some embodiments, an apelin analogue comprises sequence that is at least 80% identical to SEQ ID NO: 28. In some embodiments, an apelin analogue comprises sequence that is at least 82% identical to SEQ ID NO: 28. In some embodiments, an apelin analogue comprises sequence that is at least 84% identical to SEQ ID NO: 28. In some embodiments, an apelin analogue comprises sequence that is at least 86% identical to SEQ ID NO: 28. In some embodiments, an apelin analogue comprises sequence that is at least 88% identical to SEQ ID NO: 28. In some embodiments, an apelin analogue comprises sequence that is at least 90% identical to SEQ ID NO: 28. In some embodiments, an apelin analogue comprises sequence that is at least 92% identical to SEQ ID NO: 28. In some embodiments, an apelin analogue comprises sequence that is at least 95% identical to SEQ ID NO: 28. In some embodiments, an apelin analogue comprises sequence that is at least 90% identical to SEQ ID NO: 28. In some embodiments, an apelin analogue comprises sequence that is at least 96% identical to SEQ ID NO: 28. In some embodiments, an apelin analogue comprises sequence that is at least 97% identical to SEQ ID NO: 28. In some embodiments, an apelin analogue comprises sequence that is at least 98% identical to SEQ ID NO: 28. In some embodiments, an apelin analogue comprises sequence that is at least 99% identical to SEQ ID NO: 28.

In some embodiments, an apelin analogue comprises sequence that is at least 80% identical to SEQ ID NO: 29. In some embodiments, an apelin analogue comprises sequence that is at least 82% identical to SEQ ID NO: 29. In some embodiments, an apelin analogue comprises sequence that is at least 84% identical to SEQ ID NO: 29. In some embodiments, an apelin analogue comprises sequence that is at least 86% identical to SEQ ID NO: 29. In some embodiments, an apelin analogue comprises sequence that is at least 88% identical to SEQ ID NO: 29. In some embodiments, an apelin analogue comprises sequence that is at least 90% identical to SEQ ID NO: 29. In some embodiments, an apelin analogue comprises sequence that is at least 92% identical to SEQ ID NO: 29. In some embodiments, an apelin analogue comprises sequence that is at least 95% identical to SEQ ID NO: 29. In some embodiments, an apelin analogue comprises sequence that is at least 90% identical to SEQ ID NO: 29. In some embodiments, an apelin analogue comprises sequence that is at least 96% identical to SEQ ID NO: 29. In some embodiments, an apelin analogue comprises sequence that is at least 97% identical to SEQ ID NO: 29. In some embodiments, an apelin analogue comprises sequence that is at least 98% identical to SEQ ID NO: 29. In some embodiments, an apelin analogue comprises sequence that is at least 99% identical to SEQ ID NO: 29.

In some embodiments, an apelin analogue comprises sequence that is at least 80% identical to SEQ ID NO: 36. In some embodiments, an apelin analogue comprises sequence that is at least 82% identical to SEQ ID NO: 36. In some embodiments, an apelin analogue comprises sequence that is at least 84% identical to SEQ ID NO: 36. In some embodiments, an apelin analogue comprises sequence that is at least 86% identical to SEQ ID NO: 36. In some embodiments, an apelin analogue comprises sequence that is at least 88% identical to SEQ ID NO: 36. In some embodiments, an apelin analogue comprises sequence that is at least 90% identical to SEQ ID NO: 36. In some embodiments, an apelin analogue comprises sequence that is at least 92% identical to SEQ ID NO: 36. In some embodiments, an apelin analogue comprises sequence that is at least 95% identical to SEQ ID NO: 36. In some embodiments, an apelin analogue comprises sequence that is at least 90% identical to SEQ ID NO: 36. In some embodiments, an apelin analogue comprises sequence that is at least 96% identical to SEQ ID NO: 36. In some embodiments, an apelin analogue comprises sequence that is at least 97% identical to SEQ ID NO: 36. In some embodiments, an apelin analogue comprises sequence that is at least 98% identical to SEQ ID NO: 36. In some embodiments, an apelin analogue comprises sequence that is at least 99% identical to SEQ ID NO: 36.

In some embodiments, an apelin analogue comprises sequence that is at least 80% identical to SEQ ID NO: 37. In some embodiments, an apelin analogue comprises sequence that is at least 82% identical to SEQ ID NO: 37. In some embodiments, an apelin analogue comprises sequence that is at least 84% identical to SEQ ID NO: 37. In some embodiments, an apelin analogue comprises sequence that is at least 86% identical to SEQ ID NO: 37. In some embodiments, an apelin analogue comprises sequence that is at least 88% identical to SEQ ID NO: 37. In some embodiments, an apelin analogue comprises sequence that is at least 90% identical to SEQ ID NO: 37. In some embodiments, an apelin analogue comprises sequence that is at least 92% identical to SEQ ID NO: 37. In some embodiments, an apelin analogue comprises sequence that is at least 95% identical to SEQ ID NO: 37. In some embodiments, an apelin analogue comprises sequence that is at least 90% identical to SEQ ID NO: 37. In some embodiments, an apelin analogue comprises sequence that is at least 96% identical to SEQ ID NO: 37. In some embodiments, an apelin analogue comprises sequence that is at least 97% identical to SEQ ID NO: 37. In some embodiments, an apelin analogue comprises sequence that is at least 98% identical to SEQ ID NO: 37. In some embodiments, an apelin analogue comprises sequence that is at least 99% identical to SEQ ID NO: 37.

In some embodiments, an apelin analogue comprises sequence that is at least 80% identical to SEQ ID NO: 38. In some embodiments, an apelin analogue comprises sequence that is at least 82% identical to SEQ ID NO: 38. In some embodiments, an apelin analogue comprises sequence that is at least 84% identical to SEQ ID NO: 38. In some embodiments, an apelin analogue comprises sequence that is at least 86% identical to SEQ ID NO: 38. In some embodiments, an apelin analogue comprises sequence that is at least 88% identical to SEQ ID NO: 38. In some embodiments, an apelin analogue comprises sequence that is at least 90% identical to SEQ ID NO: 38. In some embodiments, an apelin analogue comprises sequence that is at least 92% identical to SEQ ID NO: 38. In some embodiments, an apelin analogue comprises sequence that is at least 95% identical to SEQ ID NO: 38. In some embodiments, an apelin analogue comprises sequence that is at least 90% identical to SEQ ID NO: 38. In some embodiments, an apelin analogue comprises sequence that is at least 96% identical to SEQ ID NO: 38. In some embodiments, an apelin analogue comprises sequence that is at least 97% identical to SEQ ID NO: 38. In some embodiments, an apelin analogue comprises sequence that is at least 98% identical to SEQ ID NO: 38. In some embodiments, an apelin analogue comprises sequence that is at least 99% identical to SEQ ID NO: 38.

In some embodiments, an apelin analogue comprises sequence that is at least 80% identical to SEQ ID NO: 39. In some embodiments, an apelin analogue comprises sequence that is at least 82% identical to SEQ ID NO: 39. In some embodiments, an apelin analogue comprises sequence that is at least 84% identical to SEQ ID NO: 39. In some embodiments, an apelin analogue comprises sequence that is at least 86% identical to SEQ ID NO: 39. In some embodiments, an apelin analogue comprises sequence that is at least 88% identical to SEQ ID NO: 39. In some embodiments, an apelin analogue comprises sequence that is at least 90% identical to SEQ ID NO: 39. In some embodiments, an apelin analogue comprises sequence that is at least 92% identical to SEQ ID NO: 39. In some embodiments, an apelin analogue comprises sequence that is at least 95% identical to SEQ ID NO: 39. In some embodiments, an apelin analogue comprises sequence that is at least 90% identical to SEQ ID NO: 39. In some embodiments, an apelin analogue comprises sequence that is at least 96% identical to SEQ ID NO: 39. In some embodiments, an apelin analogue comprises sequence that is at least 97% identical to SEQ ID NO: 9. In some embodiments, an apelin analogue comprises sequence that is at least 98% identical to SEQ ID NO: 39. In some embodiments, an apelin analogue comprises sequence that is at least 99% identical to SEQ ID NO: 39.

In some embodiments, an apelin analogue comprises sequence that is at least 80% identical to SEQ ID NO: 40. In some embodiments, an apelin analogue comprises sequence that is at least 82% identical to SEQ ID NO: 40. In some embodiments, an apelin analogue comprises sequence that is at least 84% identical to SEQ ID NO: 40. In some embodiments, an apelin analogue comprises sequence that is at least 86% identical to SEQ ID NO: 40. In some embodiments, an apelin analogue comprises sequence that is at least 88% identical to SEQ ID NO: 40. In some embodiments, an apelin analogue comprises sequence that is at least 90% identical to SEQ ID NO: 40. In some embodiments, an apelin analogue comprises sequence that is at least 92% identical to SEQ ID NO: 0. In some embodiments, an apelin analogue comprises sequence that is at least 95% identical to SEQ ID NO: 40. In some embodiments, an apelin analogue comprises sequence that is at least 90% identical to SEQ ID NO: 40. In some embodiments, an apelin analogue comprises sequence that is at least 96% identical to SEQ ID NO: 40. In some embodiments, an apelin analogue comprises sequence that is at least 97% identical to SEQ ID NO: 40. In some embodiments, an apelin analogue comprises sequence that is at least 98% identical to SEQ ID NO: 40. In some embodiments, an apelin analogue comprises sequence that is at least 99% identical to SEQ ID NO: 40.

In some embodiments, an apelin analogue comprises sequence that is at least 80% identical to SEQ ID NO: 41. In some embodiments, an apelin analogue comprises sequence that is at least 82% identical to SEQ ID NO: 41. In some embodiments, an apelin analogue comprises sequence that is at least 84% identical to SEQ ID NO: 41. In some embodiments, an apelin analogue comprises sequence that is at least 86% identical to SEQ ID NO: 41. In some embodiments, an apelin analogue comprises sequence that is at least 88% identical to SEQ ID NO: 41. In some embodiments, an apelin analogue comprises sequence that is at least 90% identical to SEQ ID NO: 41. In some embodiments, an apelin analogue comprises sequence that is at least 92% identical to SEQ ID NO: 41. In some embodiments, an apelin analogue comprises sequence that is at least 95% identical to SEQ ID NO: 41. In some embodiments, an apelin analogue comprises sequence that is at least 90% identical to SEQ ID NO: 41. In some embodiments, an apelin analogue comprises sequence that is at least 96% identical to SEQ ID NO: 41. In some embodiments, an apelin analogue comprises sequence that is at least 97% identical to SEQ ID NO: 41. In some embodiments, an apelin analogue comprises sequence that is at least 98% identical to SEQ ID NO: 41. In some embodiments, an apelin analogue comprises sequence that is at least 99% identical to SEQ ID NO: 41.

In some embodiments, an apelin analogue comprises sequence that is at least 80% identical to SEQ ID NO: 56. In some embodiments, an apelin analogue comprises sequence that is at least 82% identical to SEQ ID NO: 56. In some embodiments, an apelin analogue comprises sequence that is at least 84% identical to SEQ ID NO: 56. In some embodiments, an apelin analogue comprises sequence that is at least 86% identical to SEQ ID NO: 56. In some embodiments, an apelin analogue comprises sequence that is at least 88% identical to SEQ ID NO: 56. In some embodiments, an apelin analogue comprises sequence that is at least 90% identical to SEQ ID NO: 56. In some embodiments, an apelin analogue comprises sequence that is at least 92% identical to SEQ ID NO: 56. In some embodiments, an apelin analogue comprises sequence that is at least 95% identical to SEQ ID NO: 56. In some embodiments, an apelin analogue comprises sequence that is at least 90% identical to SEQ ID NO: 56. In some embodiments, an apelin analogue comprises sequence that is at least 96% identical to SEQ ID NO: 56. In some embodiments, an apelin analogue comprises sequence that is at least 97% identical to SEQ ID NO: 56. In some embodiments, an apelin analogue comprises sequence that is at least 98% identical to SEQ ID NO: 56. In some embodiments, an apelin analogue comprises sequence that is at least 99% identical to SEQ ID NO: 56.

In some embodiments, an apelin analogue comprises sequence that is at least 80% identical to SEQ ID NO: 57. In some embodiments, an apelin analogue comprises sequence that is at least 82% identical to SEQ ID NO: 57. In some embodiments, an apelin analogue comprises sequence that is at least 84% identical to SEQ ID NO: 57. In some embodiments, an apelin analogue comprises sequence that is at least 86% identical to SEQ ID NO: 57. In some embodiments, an apelin analogue comprises sequence that is at least 88% identical to SEQ ID NO: 57. In some embodiments, an apelin analogue comprises sequence that is at least 90% identical to SEQ ID NO: 57. In some embodiments, an apelin analogue comprises sequence that is at least 92% identical to SEQ ID NO: 57. In some embodiments, an apelin analogue comprises sequence that is at least 95% identical to SEQ ID NO: 57. In some embodiments, an apelin analogue comprises sequence that is at least 90% identical to SEQ ID NO: 57. In some embodiments, an apelin analogue comprises sequence that is at least 96% identical to SEQ ID NO: 57. In some embodiments, an apelin analogue comprises sequence that is at least 97% identical to SEQ ID NO: 57. In some embodiments, an apelin analogue comprises sequence that is at least 98% identical to SEQ ID NO: 57. In some embodiments, an apelin analogue comprises sequence that is at least 99% identical to SEQ ID NO: 57.

In some embodiments, an apelin analogue comprises sequence that is at least 80% identical to SEQ ID NO: 58. In some embodiments, an apelin analogue comprises sequence that is at least 82% identical to SEQ ID NO: 58. In some embodiments, an apelin analogue comprises sequence that is at least 84% identical to SEQ ID NO: 58. In some embodiments, an apelin analogue comprises sequence that is at least 86% identical to SEQ ID NO: 58. In some embodiments, an apelin analogue comprises sequence that is at least 88% identical to SEQ ID NO: 58. In some embodiments, an apelin analogue comprises sequence that is at least 90% identical to SEQ ID NO: 58. In some embodiments, an apelin analogue comprises sequence that is at least 92% identical to SEQ ID NO: 58. In some embodiments, an apelin analogue comprises sequence that is at least 95% identical to SEQ ID NO: 58. In some embodiments, an apelin analogue comprises sequence that is at least 90% identical to SEQ ID NO: 58. In some embodiments, an apelin analogue comprises sequence that is at least 96% identical to SEQ ID NO: 58. In some embodiments, an apelin analogue comprises sequence that is at least 97% identical to SEQ ID NO: 58. In some embodiments, an apelin analogue comprises sequence that is at least 98% identical to SEQ ID NO: 58. In some embodiments, an apelin analogue comprises sequence that is at least 99% identical to SEQ ID NO: 58.

In some embodiments, an apelin analogue comprises sequence that is at least 80% identical to SEQ ID NO: 59. In some embodiments, an apelin analogue comprises sequence that is at least 82% identical to SEQ ID NO: 59. In some embodiments, an apelin analogue comprises sequence that is at least 84% identical to SEQ ID NO: 59. In some embodiments, an apelin analogue comprises sequence that is at least 86% identical to SEQ ID NO: 59. In some embodiments, an apelin analogue comprises sequence that is at least 88% identical to SEQ ID NO: 59. In some embodiments, an apelin analogue comprises sequence that is at least 90% identical to SEQ ID NO: 59. In some embodiments, an apelin analogue comprises sequence that is at least 92% identical to SEQ ID NO: 59. In some embodiments, an apelin analogue comprises sequence that is at least 95% identical to SEQ ID NO: 59. In some embodiments, an apelin analogue comprises sequence that is at least 90% identical to SEQ ID NO: 59. In some embodiments, an apelin analogue comprises sequence that is at least 96% identical to SEQ ID NO: 59. In some embodiments, an apelin analogue comprises sequence that is at least 97% identical to SEQ ID NO: 59. In some embodiments, an apelin analogue comprises sequence that is at least 98% identical to SEQ ID NO: 59. In some embodiments, an apelin analogue comprises sequence that is at least 99% identical to SEQ ID NO: 59.

In some embodiments, an apelin analogue comprises sequence that is at least 80% identical to SEQ ID NO: 60. In some embodiments, an apelin analogue comprises sequence that is at least 82% identical to SEQ ID NO: 60. In some embodiments, an apelin analogue comprises sequence that is at least 84% identical to SEQ ID NO: 60. In some embodiments, an apelin analogue comprises sequence that is at least 86% identical to SEQ ID NO: 60. In some embodiments, an apelin analogue comprises sequence that is at least 88% identical to SEQ ID NO: 60. In some embodiments, an apelin analogue comprises sequence that is at least 90% identical to SEQ ID NO: 60. In some embodiments, an apelin analogue comprises sequence that is at least 92% identical to SEQ ID NO: 60. In some embodiments, an apelin analogue comprises sequence that is at least 95% identical to SEQ ID NO: 60. In some embodiments, an apelin analogue comprises sequence that is at least 90% identical to SEQ ID NO: 60. In some embodiments, an apelin analogue comprises sequence that is at least 96% identical to SEQ ID NO: 60. In some embodiments, an apelin analogue comprises sequence that is at least 97% identical to SEQ ID NO: 60. In some embodiments, an apelin analogue comprises sequence that is at least 98% identical to SEQ ID NO: 60. In some embodiments, an apelin analogue comprises sequence that is at least 99% identical to SEQ ID NO: 60.

In some embodiments, an apelin analogue comprises sequence that is at least 80% identical to SEQ ID NO: 61. In some embodiments, an apelin analogue comprises sequence that is at least 82% identical to SEQ ID NO: 61. In some embodiments, an apelin analogue comprises sequence that is at least 84% identical to SEQ ID NO: 61. In some embodiments, an apelin analogue comprises sequence that is at least 86% identical to SEQ ID NO: 61. In some embodiments, an apelin analogue comprises sequence that is at least 88% identical to SEQ ID NO: 61. In some embodiments, an apelin analogue comprises sequence that is at least 90% identical to SEQ ID NO: 61. In some embodiments, an apelin analogue comprises sequence that is at least 92% identical to SEQ ID NO: 61. In some embodiments, an apelin analogue comprises sequence that is at least 95% identical to SEQ ID NO: 61. In some embodiments, an apelin analogue comprises sequence that is at least 90% identical to SEQ ID NO: 61. In some embodiments, an apelin analogue comprises sequence that is at least 96% identical to SEQ ID NO: 61. In some embodiments, an apelin analogue comprises sequence that is at least 97% identical to SEQ ID NO: 61. In some embodiments, an apelin analogue comprises sequence that is at least 98% identical to SEQ ID NO: 61. In some embodiments, an apelin analogue comprises sequence that is at least 99% identical to SEQ ID NO: 61.

### Leader Sequence

In some embodiments, an apelin receptor agonist comprises a leader sequence. In some embodiments, an apelin receptor agonist comprises a leader sequence at the N-terminus. In some embodiments, an apelin receptor agonist comprises a leader sequence at the N-terminus of a carrier moiety. In some embodiments, an apelin receptor agonist comprises a leader sequence at the N-terminus of an Fc domain. In some embodiments, a leader sequence is cleaved prior to secretion.

In some embodiments, a leader sequence comprises an osteonectin leader sequence. In some embodiments, a leader sequence comprises SEQ ID NO: 62 (MRAWIFFLLCLAGRALA). In some embodiments, a leader sequence comprises an amino acid sequence that is at least 80% identical to SEQ ID NO: 62. In some embodiments, a leader sequence comprises an amino acid sequence that is at least 85% identical to SEQ ID NO: 62. In some embodiments, a leader sequence comprises an amino acid sequence that is at least 90% identical to SEQ ID NO: 62. In some embodiments, a leader sequence comprises an amino acid sequence that is at least 92% identical to SEQ ID NO: 62. In some embodiments, a leader sequence comprises an amino acid sequence that is at least 95% identical to SEQ ID NO: 62. In some embodiments, a leader sequence comprises an amino acid sequence that is at least 98% identical to SEQ ID NO: 62. In some embodiments, a leader sequence comprises an amino acid sequence that is at least 99% identical to SEQ ID NO: 62.

### Properties of Apelin Receptor Agonists

The present application provides among other things, apelin receptor agonists comprising an apelin analogue or an elabela analogue, which have unexpectedly superior properties, including, but not limited to improved signaling properties, higher half-life, and reduced desensitization as compared to wild-type apelin or elabela. In some embodiments, apelin receptor agonists of the present invention bind APJ with higher affinity and specificity as compared to wild-type apelin agonists.

### Improved Signaling in APJ Pathways

Apelin receptor agonists of the present invention have improved signaling properties as compared to wild-type or endogenous apelin or elabela.

In some embodiments, an apelin receptor agonist has a bias for Gα signaling over β-arrestin signaling. In some embodiments, an apelin receptor agonist has a bias for Ga signaling over β-arrestin signaling by at least about 1.5-fold. In some embodiments, an apelin receptor agonist has a bias for Gα signaling over β-arrestin signaling by at least about 2-fold. In some embodiments, an apelin receptor agonist has a bias for Gα signaling over β-arrestin signaling by at least about 2.5-fold. In some embodiments, an apelin receptor agonist has a bias for Gα signaling over β-arrestin signaling by at least about 3-fold. In some embodiments, an apelin receptor agonist has a bias for Gα signaling over β-arrestin signaling by at least about 4-fold. In some embodiments, an apelin receptor agonist has a bias for Gα signaling over β-arrestin signaling by at least about 5-fold. In some embodiments, an apelin receptor agonist has a bias for Gα signaling over β-arrestin signaling by at least about 7-fold. In some embodiments, an apelin receptor agonist has a bias for Gα signaling over β-arrestin signaling by at least about 10-fold. In some embodiments, an apelin receptor agonist has a bias for Gα signaling over β-arrestin signaling by at least about 12-fold. In some embodiments, an apelin receptor agonist has a bias for Gα signaling over β-arrestin signaling by at least about 15-fold. In some embodiments, an apelin receptor agonist has a bias for Gα signaling over β-arrestin signaling by at least about 20-fold. In some embodiments, an apelin receptor agonist has a bias for Gα signaling over β-arrestin signaling by at least about 25-fold. In some embodiments, an apelin receptor agonist has a bias for Gα signaling over β-arrestin signaling by at least about 30-fold. In some embodiments, an apelin receptor agonist has a bias for Gα signaling over β-arrestin signaling by at least about 40-fold. In some embodiments, an apelin receptor agonist has a bias for Gα signaling over β-arrestin signaling by at least about 50-fold. In some embodiments, an apelin receptor agonist has a bias for Gα signaling over β-arrestin signaling by at least about 50-fold. In some embodiments, an apelin receptor agonist has a bias for Gα signaling over β-arrestin signaling by at least about 100-fold. In some embodiments, an apelin receptor agonist has a bias for Gα signaling over β-arrestin signaling by at least about 200-fold. In some embodiments, an apelin receptor agonist has a bias for Gα signaling over β-arrestin signaling by at least about 500-fold.

In some embodiments, an apelin receptor agonist has a G_{αi} signaling assay EC50 value of less than 60 nM as measured by the depletion of cAMP. In some embodiments, an apelin receptor agonist has a G_{αi} signaling assay EC50 value of less than 80 nM as measured by the depletion of cAMP. In some embodiments, an apelin receptor agonist has a G_{αi} signaling assay EC50 value of less than 75 nM as measured by the depletion of cAMP. In some embodiments, an apelin receptor agonist has a G_{αi} signaling assay EC50 value of less than 70 nM as measured by the depletion of cAMP. In some embodiments, an apelin receptor agonist has a G_{αi} signaling assay EC50 value of less than 65 nM as measured by the depletion of cAMP. In some embodiments, an apelin receptor agonist has a G_{αi} signaling assay EC50 value of less than 60 nM as measured by the depletion of cAMP. In some embodiments, an apelin receptor agonist has a G_{αi} signaling assay EC50 value of less than 55 nM as measured by the depletion of cAMP. In some embodiments, an apelin receptor agonist has a G_{αi} signaling assay EC50 value of less than 50 nM as measured by the depletion of cAMP. In some embodiments, an apelin receptor agonist has a G_{αi} signaling assay EC50 value of less than 45 nM as measured by the depletion of cAMP. In some embodiments, an apelin receptor agonist has a G_{αi} signaling assay EC50 value of less than 40 nM as measured by the depletion of cAMP. In some embodiments, an apelin receptor agonist has a G_{αi} signaling assay EC50 value of less than 35 nM as measured by the depletion of cAMP. In some embodiments, an apelin receptor agonist has a G_{αi} signaling assay EC50 value of less than 30 nM as measured by the depletion of cAMP. In some embodiments, an apelin receptor agonist has a G_{αi} signaling assay EC50 value of less than 25 nM as measured by the depletion of cAMP. In some embodiments, an apelin receptor agonist has a G_{αi} signaling assay EC50 value of less than 20 nM as measured by the depletion of cAMP. In some embodiments, an apelin receptor agonist has a G_{αi} signaling assay EC50 value of less than 15 nM as measured by the depletion of cAMP. In some embodiments, an apelin receptor agonist has a G_{αi} signaling assay EC50 value of less than 10 nM as measured by the depletion of cAMP. In some embodiments, an apelin receptor agonist has a G_{αi} signaling assay EC50 value of less than 5 nM as measured by the depletion of cAMP. In some embodiments, an apelin receptor agonist has a G_{αi} signaling assay EC50 value of less than 2 nM as measured by the depletion of cAMP. In some embodiments, an apelin receptor agonist has a G_{αi} signaling assay EC50 value of less than 1 nM as measured by the depletion of cAMP.

In some embodiments, an apelin receptor agonist has beta-Arrestin signaling assay EC50 value of less than 1000 nM. In some embodiments, an apelin receptor agonist has beta-Arrestin signaling assay EC50 value of less than 600 nM. In some embodiments, an apelin receptor agonist has beta-Arrestin signaling assay EC50 value of less than 500 nM. In some embodiments, an apelin receptor agonist has beta-Arrestin signaling assay EC50 value of less than 450 nM. In some embodiments, an apelin receptor agonist has beta-Arrestin signaling assay EC50 value of less than 400 nM. In some embodiments, an apelin receptor agonist has beta-Arrestin signaling assay EC50 value of less than 350 nM. In some embodiments, an apelin receptor agonist has beta-Arrestin signaling assay EC50 value of less than 300 nM. In some embodiments, an apelin receptor agonist has beta-Arrestin signaling assay EC50 value of less than 250 nM. In some embodiments, an apelin receptor agonist has beta-Arrestin signaling assay EC50 value of less than 200 nM. In some embodiments, an apelin receptor agonist has beta-Arrestin signaling assay EC50 value of less than 150 nM. In some embodiments, an apelin receptor agonist has beta-Arrestin signaling assay EC50 value of less than 100 nM. In some embodiments, an apelin receptor agonist has beta-Arrestin signaling assay EC50 value of less than 80 nM. In some embodiments, an apelin receptor agonist has beta-Arrestin signaling assay EC50 value of less than 60 nM. In some embodiments, an apelin receptor agonist has beta-Arrestin signaling assay EC50 value of less than 50 nM. In some embodiments, an apelin receptor agonist has beta-Arrestin signaling assay EC50 value of less than 40 nM. In some embodiments, an apelin receptor agonist has beta-Arrestin signaling assay EC50 value of less than 30 nM. In some embodiments, an apelin receptor agonist has beta-Arrestin signaling assay EC50 value of less than 20 nM. In some embodiments, an apelin receptor agonist has beta-Arrestin signaling assay EC50 value of less than 10 nM. In some embodiments, an apelin receptor agonist has beta-Arrestin signaling assay EC50 value of less than 5 nM.

In some embodiments, an apelin receptor agonist has beta-Arrestin signaling assay EC50 value of greater than 5 nM. In some embodiments, an apelin receptor agonist has beta-Arrestin signaling assay EC50 value of greater than 10 nM. In some embodiments, an apelin receptor agonist has beta-Arrestin signaling assay EC50 value of greater than 20 nM. In some embodiments, an apelin receptor agonist has beta-Arrestin signaling assay EC50 value of greater than 30 nM. In some embodiments, an apelin receptor agonist has beta-Arrestin signaling assay EC50 value of greater than 40 nM. In some embodiments, an apelin receptor agonist has beta-Arrestin signaling assay EC50 value of greater than 50 nM. In some embodiments, an apelin receptor agonist has beta-Arrestin signaling assay EC50 value of greater than 60 nM. In some embodiments, an apelin receptor agonist has beta-Arrestin signaling assay EC50 value of greater than 70 nM. In some embodiments, an apelin receptor agonist has beta-Arrestin signaling assay EC50 value of greater than 80 nM. In some embodiments, an apelin receptor agonist has beta-Arrestin signaling assay EC50 value of greater than 90 nM. In some embodiments, an apelin receptor agonist has beta-Arrestin signaling assay EC50 value of greater than 100 nM. In some embodiments, an apelin receptor agonist has beta-Arrestin signaling assay EC50 value of greater than 200 nM. In some embodiments, an apelin receptor agonist has beta-Arrestin signaling assay EC50 value of greater than 300 nM. In some embodiments, an apelin receptor agonist has beta-Arrestin signaling assay EC50 value of greater than 400 nM. In some embodiments, an apelin receptor agonist has beta-Arrestin signaling assay EC50 value of greater than 500 nM. In some embodiments, an apelin receptor agonist has beta-Arrestin signaling assay EC50 value of greater than 600 nM. In some embodiments, an apelin receptor agonist has beta-Arrestin signaling assay EC50 value of greater than 700 nM. In some embodiments, an apelin receptor agonist has beta-Arrestin signaling assay EC50 value of greater than 800 nM. In some embodiments, an apelin receptor agonist has beta-Arrestin signaling assay EC50 value of greater than 900 nM. In some embodiments, an apelin receptor agonist has beta-Arrestin signaling assay EC50 value of greater than 1000 nM. In some embodiments, an apelin receptor agonist has beta-Arrestin signaling assay EC50 value of greater than 2000 nM. In some embodiments, an apelin receptor agonist has beta-Arrestin signaling assay EC50 value of greater than 5000 nM.

In some embodiments, an apelin receptor agonist has an ERK activation assay EC50 value of less than 50 nM. In some embodiments, an apelin receptor agonist has an ERK activation assay EC50 value of less than 40 nM. In some embodiments, an apelin receptor agonist has an ERK activation assay EC50 value of less than 30 nM. In some embodiments, an apelin receptor agonist has an ERK activation assay EC50 value of less than 25 nM. In some embodiments, an apelin receptor agonist has an ERK activation assay EC50 value of less than 20 nM. In some embodiments, an apelin receptor agonist has an ERK activation assay EC50 value of less than 15 nM. In some embodiments, an apelin receptor agonist has an ERK activation assay EC50 value of less than 10 nM. In some embodiments, an apelin receptor agonist has an ERK activation assay EC50 value of less than 5 nM. In some embodiments, an apelin receptor agonist has an ERK activation assay EC50 value of less than 2 nM. In some embodiments, an apelin receptor agonist has an ERK activation assay EC50 value of less than 1 nM.

### Half-Life

In some embodiments, apelin receptor agonists of the present invention have higher efficacy and potency as compared to a wild-type or endogenous apelin or elabela. In some embodiments, apelin receptor agonists of the present invention have increased half-lives as compared to a wild-type or endogenous apelin or elabela. In some embodiments, apelin receptor agonists of the present invention have reduced susceptibility to proteolytic cleavage as compared to a wild-type or endogenous apelin or elabela. In some embodiments, apelin receptor agonists of the present invention have reduced susceptibility to renal clearance as compared to a wild-type or endogenous apelin or elabela.

In some embodiments, an apelin receptor agonist has a plasma or serum in vivo half-life of at least about 30 minutes. In some embodiments, an apelin receptor agonist has a plasma or serum in vivo half-life of at least about 1 hour. In some embodiments, an apelin receptor agonist has a plasma or serum in vivo half-life of at least about 2 hours. In some embodiments, an apelin receptor agonist has a plasma or serum in vivo half-life of at least about 3 hours. In some embodiments, an apelin receptor agonist has a plasma or serum in vivo half-life of at least about 4 hours. In some embodiments, an apelin receptor agonist has a plasma or serum in vivo half-life of at least about 5 hours. In some embodiments, an apelin receptor agonist has a plasma or serum in vivo half-life of at least about 6 hours. In some embodiments, an apelin receptor agonist has a plasma or serum in vivo half-life of at least about 7 hours. In some embodiments, an apelin receptor agonist has a plasma or serum in vivo half-life of at least about 8 hours. In some embodiments, an apelin receptor agonist has a plasma or serum in vivo half-life of at least about 9 hours. In some embodiments, an apelin receptor agonist has a plasma or serum in vivo half-life of at least about 10 hours. In some embodiments, an apelin receptor agonist has a plasma or serum in vivo half-life of at least about 12 hours. In some embodiments, an apelin receptor agonist has a plasma or serum in vivo half-life of at least about 24 hours. In some embodiments, an apelin receptor agonist has a plasma or serum in vivo half-life of at least about 2 days. In some embodiments, an apelin receptor agonist has a plasma or serum in vivo half-life of at least about 3 days. In some embodiments, an apelin receptor agonist has a plasma or serum in vivo half-life of at least about 4 days. In some embodiments, an apelin receptor agonist has a plasma or serum in vivo half-life of at least about 5 days. In some embodiments, an apelin receptor agonist has a plasma or serum in vivo half-life of at least about 6 days. In some embodiments, an apelin receptor agonist has a plasma or serum in vivo half-life of at least about 7 days. In some embodiments, an apelin receptor agonist has a plasma or serum in vivo half-life of at least about 10 days. In some embodiments, an apelin receptor agonist has a plasma or serum in vivo half-life of at least about 15 days. In some embodiments, an apelin receptor agonist has a plasma or serum in vivo half-life of at least about two weeks. In some embodiments, an apelin receptor agonist has a plasma or serum in vivo half-life of at least about 3 weeks. In some embodiments, an apelin receptor agonist has a plasma or serum in vivo half-life of at least about 4 weeks.

In some embodiments, an apelin receptor agonist has a half-life that is at least 2-fold greater than the half-life of a wild-type apelin or elabela. In some embodiments, an apelin receptor agonist has a half-life that is at least 3-fold greater than the half-life of a wild-type apelin or elabela. In some embodiments, an apelin receptor agonist has a half-life that is at least 4-fold greater than the half-life of a wild-type apelin or elabela. In some embodiments, an apelin receptor agonist has a half-life that is at least 5-fold greater than the half-life of a wild-type apelin or elabela. In some embodiments, an apelin receptor agonist has a half-life that is at least 6-fold greater than the half-life of a wild-type apelin or elabela. In some embodiments, an apelin receptor agonist has a half-life that is at least 7-fold greater than the half-life of a wild-type apelin or elabela. In some embodiments, an apelin receptor agonist has a half-life that is at least 8-fold greater than the half-life of a wild-type apelin or elabela. In some embodiments, an apelin receptor agonist has a half-life that is at least 9-fold greater than the half-life of a wild-type apelin or elabela. In some embodiments, an apelin receptor agonist has a half-life that is at least 10-fold greater than the half-life of a wild-type apelin or elabela. In some embodiments, an apelin receptor agonist has a half-life that is at least 11-fold greater than the half-life of a wild-type apelin or elabela. In some embodiments, an apelin receptor agonist has a half-life that is at least 12-fold greater than the half-life of a wild-type apelin or elabela. In some embodiments, an apelin receptor agonist has a half-life that is at least 15-fold greater than the half-life of a wild-type apelin or elabela. In some embodiments, an apelin receptor agonist has a half-life that is at least 20-fold greater than the half-life of a wild-type apelin or elabela. In some embodiments, an apelin receptor agonist has a half-life that is at least 25-fold greater than the half-life of a wild-type apelin or elabela. In some embodiments, an apelin receptor agonist has a half-life that is at least 30-fold greater than the half-life of a wild-type apelin or elabela. In some embodiments, an apelin receptor agonist has a half-life that is at least 40-fold greater than the half-life of a wild-type apelin or elabela. In some embodiments, an apelin receptor agonist has a half-life that is at least 50-fold greater than the half-life of a wild-type apelin or elabela. In some embodiments, an apelin receptor agonist has a half-life that is at least 100-fold greater than the half-life of a wild-type apelin or elabela.

### Desensitization

GPCR desensitization is a multi-prong problem that takes place over multiple phases. In general, β-arrestin signaling has been associated with desensitization, but these effects have not been extensively studied with APJ.

GPCR desensitization can occur in both short-term and long-term phases. In the short-term, the receptor can be internalized into endosomes, thus removing them from the cell membrane and preventing interactions with the natural ligands or drugs. While the internalized receptors can sometimes be recycled to the membrane, making them available once again, in other circumstances, the endosomes will fuse with lysosomes and target the receptor for degradation, leading to longer-term desensitization.

In the longer term, persistent signaling can lead to transcriptional downregulation of the receptor and its downstream effectors. This causes the removal of the receptor from the cell membrane or the decoupling of receptor activation to downstream effector functions, respectively, once again decreasing the effectiveness of both the natural ligands and drugs.

In some embodiments, apelin receptor agonists of the present invention have reduced desensitization as compared to a wild-type or endogenous apelin or elabela.

In some embodiments, an apelin receptor agonist causes a reduced desensitization that is at least 2-fold less than the desensitization of a wild-type apelin or elabela. In some embodiments, an apelin receptor agonist causes a reduced desensitization that is at least 3-fold less than the desensitization of a wild-type apelin or elabela. In some embodiments, an apelin receptor agonist causes a reduced desensitization that is at least 4-fold less than the desensitization of a wild-type apelin or elabela. In some embodiments, an apelin receptor agonist causes a reduced desensitization that is at least 5-fold less than the desensitization of a wild-type apelin or elabela. In some embodiments, an apelin receptor agonist causes a reduced desensitization that is at least 6-fold less than the desensitization of a wild-type apelin or elabela. In some embodiments, an apelin receptor agonist causes a reduced desensitization that is at least 7-fold less than the desensitization of a wild-type apelin or elabela. In some embodiments, an apelin receptor agonist causes a reduced desensitization that is at least 8-fold less than the desensitization of a wild-type apelin or elabela. In some embodiments, an apelin receptor agonist causes a reduced desensitization that is at least 9-fold less than the desensitization of a wild-type apelin or elabela. In some embodiments, an apelin receptor agonist causes a reduced desensitization that is at least 10-fold less than the desensitization of a wild-type apelin or elabela. In some embodiments, an apelin receptor agonist causes a reduced desensitization that is at least 12-fold less than the desensitization of a wild-type apelin or elabela. In some embodiments, an apelin receptor agonist causes a reduced desensitization that is at least 15-fold less than the desensitization of a wild-type apelin or elabela. In some embodiments, an apelin receptor agonist causes a reduced desensitization that is at least 20-fold less than the desensitization of a wild-type apelin or elabela. In some embodiments, an apelin receptor agonist causes a reduced desensitization that is at least 30-fold less than the desensitization of a wild-type apelin or elabela. In some embodiments, an apelin receptor agonist causes a reduced desensitization that is at least 40-fold less than the desensitization of a wild-type apelin or elabela. In some embodiments, an apelin receptor agonist causes a reduced desensitization that is at least 50-fold less than the desensitization of a wild-type apelin or elabela. In some embodiments, an apelin receptor agonist causes a reduced desensitization that is at least 60-fold less than the desensitization of a wild-type apelin or elabela. In some embodiments, an apelin receptor agonist causes a reduced desensitization that is at least 80-fold less than the desensitization of a wild-type apelin or elabela. In some embodiments, an apelin receptor agonist causes a reduced desensitization that is at least 100-fold less than the desensitization of a wild-type apelin or elabela.

### Internalization

In some embodiments, apelin receptor agonists of the present invention have reduced internalization as compared to a wild-type or endogenous apelin or elabela. In some embodiments, apelin receptor agonists of the present invention are substantially free of internalization. The reduced internalization rate of the apelin receptor agonist compared to the wild-type ligands may be a desirable property, as it could allow for more efficient and sustained activation of the apelin receptor and its downstream effects.

In some embodiments, an apelin receptor agonist has reduced internalization that is at least 2-fold less than the internalization of a wild-type apelin or elabela. In some embodiments, an apelin receptor agonist has reduced internalization that is at least 3-fold less than the internalization of a wild-type apelin or elabela. In some embodiments, an apelin receptor agonist has reduced internalization that is at least 4-fold less than the internalization of a wild-type apelin or elabela. In some embodiments, an apelin receptor agonist has reduced internalization that is at least 5-fold less than the internalization of a wild-type apelin or elabela. In some embodiments, an apelin receptor agonist has reduced internalization that is at least 6-fold less than the internalization of a wild-type apelin or elabela. In some embodiments, an apelin receptor agonist has reduced internalization that is at least 7-fold less than the internalization of a wild-type apelin or elabela. In some embodiments, an apelin receptor agonist has reduced internalization that is at least 8-fold less than the internalization of a wild-type apelin or elabela. In some embodiments, an apelin receptor agonist has reduced internalization that is at least 9-fold less than the internalization of a wild-type apelin or elabela. In some embodiments, an apelin receptor agonist has reduced internalization that is at least 10-fold less than the internalization of a wild-type apelin or elabela.

In some embodiments, an apelin receptor agonist is internalized at a rate that is at least 2 times lower than the internalization rate of a wild-type apelin or elabela. In some embodiments, an apelin receptor agonist is internalized at a rate that is at least 3 times lower than the internalization rate of a wild-type apelin or elabela. In some embodiments, an apelin receptor agonist is internalized at a rate that is at least 4 times lower than the internalization rate of a wild-type apelin or elabela. In some embodiments, an apelin receptor agonist is internalized at a rate that is at least 5 times lower than the internalization rate of a wild-type apelin or elabela. In some embodiments, an apelin receptor agonist is internalized at a rate that is at least 6 times lower than the internalization rate of a wild-type apelin or elabela. In some embodiments, an apelin receptor agonist is internalized at a rate that is at least 7 times lower than the internalization rate of a wild-type apelin or elabela. In some embodiments, an apelin receptor agonist is internalized at a rate that is at least 8 times lower than the internalization rate of a wild-type apelin or elabela. In some embodiments, an apelin receptor agonist is internalized at a rate that is at least 9 times lower than the internalization rate of a wild-type apelin or elabela. In some embodiments, an apelin receptor agonist is internalized at a rate that is at least 10 times lower than the internalization rate of a wild-type apelin or elabela.

In some embodiments, an apelin receptor agonist is not internalized. In some embodiments, an apelin receptor agonist is internalized only when the apelin receptor agonist is present at a concentration of greater than 1000 nM. In some embodiments, an apelin receptor agonist is internalized only when the apelin receptor agonist is present at a concentration of greater than 500 nM. In some embodiments, an apelin receptor agonist is internalized only when the apelin receptor agonist is present at a concentration of greater than 100 nM. In some embodiments, an apelin receptor agonist is internalized only when the apelin receptor agonist is present at a concentration of greater than 50 nM. In some embodiments, an apelin receptor agonist is internalized only when the apelin receptor agonist is present at a concentration of greater than 100 nM.

Treatment with Apelin Receptor Agonist

The present invention provides, among other things, a method of treating a disease by administering an apelin receptor agonist disclosed in the present invention. In some embodiments, the administering of apelin receptor agonist of the present invention improves, stabilizes, or reduces one or more symptoms of the disease.

In some embodiments, the disease is implicated in apelin receptor signaling. In some embodiments, the disease is pulmonary arterial hypertension (PAH). In some embodiments, the disease is systemic sclerosis (SSc). In some embodiments, the disease is heart failure with preserved ejection fraction (HfpEf). In some embodiments, the disease is age-associated sarcopenia. In some embodiments, the disease is acute kidney injury (AKI). In some embodiments, the disease is cardiovascular disease. In some embodiments, the disease is cancer. In some embodiments, the disease is diabetes. In some embodiments, the disease is acute decompensated heart failure. In some embodiments, the disease is congestive heart failure. In some embodiments, the disease is myocardial infarction. In some embodiments, the disease is cardiomyopathy. In some embodiments, the disease is ischemia. In some embodiments, the disease is ischemia/reperfusion injury. In some embodiments, the disease is pulmonary hypertension. In some embodiments, the disease is obesity. In some embodiments, the disease is metastatic disease. In some embodiments, the disease is fluid homeostasis. In some embodiments, the disease is pathological angiogenesis. In some embodiments, the disease is retinopathy. In some embodiments, the disease is fibrosis. In some embodiments, the disease is HIV infection. In some embodiments, the disease is insulin resistance and type 2 diabetes. In some embodiments, the disease is other apelin related diseases.

The apelin receptor agonists of the invention (also referred to herein as “active compounds”) can be incorporated into pharmaceutical compositions suitable for administration. Such compositions typically comprise the apelin receptor agonists and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. F or intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. F or the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

F or administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. F or transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. F or transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention is dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### EXAMPLES

While certain compounds, compositions, and methods of the present disclosure have been described with specificity in accordance with certain embodiments, the following examples serve only to illustrate the disclosure and are not intended to limit the same.

### Example 1—Effect of Apelin Receptor Agonists on Apelin Receptor Signaling Pathways

This Example measures the effect of apelin receptor agonists of the present invention on various downstream signaling pathways of apelin receptor system.

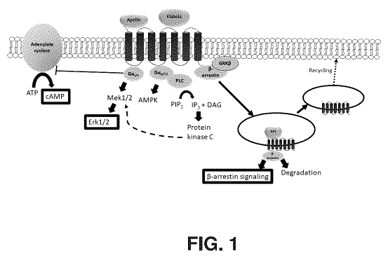

The apelin receptor couples to pertussis toxin-sensitive inhibitory G proteins (Gα_{i/o}) with subsequent activation of extracellular-regulated kinases (ERKs) signaling cascades (FIG. 1). These cascades lead to a broad range of physiological effects that are dependent on the type of cell that is activated. All putative apelin and ELA isoforms elicit canonical Gi-mediated inhibition of adenylate cyclase, which results in inhibition of cAMP. Signaling via β-arrestin leads to apelin receptor desensitization and internalization. The internalized receptor is targeted for degradation or recycled to the cell surface. β-Arrestin-mediated signaling may also contribute to the actions of apelin.

In this example, commercially available HitHunter® cAMP assay was used to determine the effect of apelin receptor agonists on Gₐᵢ signaling, measured cAMP depletion. Next, commercially available PathHunter® beta-arrestin assay was used to determine the effect of apelin receptor agonists on beta-arrestin signaling. Additionally, the effect of apelin receptor agonists on ERK signaling was determined.

cAMP Signaling Assay

In a 384-well plate (Corning #3897), cAMP Hunter™ CHO-K1 AGTRL1 Gi cells (Eurofins DiscoverX #95-0147C2) were seeded at 1×104 cells/well in 20 μL AssayComplete Cell Plating 2 Reagent (Eurofins DiscoverX #93-0563R2A) and were incubated overnight at 37° C. with 5% CO2. On the following day, the plated cells were washed with Assay Buffer (1×HBSS+10 mM HEPES) and 20 μL/well of 2-parts Assay Buffer and 1-part cAMP antibody from the HitHunter® cAMP Assay for Small Molecules Kit (Eurofins DiscoverX #90-0075SM25) was added. The plate was treated with 5 μL/well of compounds serially diluted in Assay Buffer+15 μM Forskolin (Millipore Sigma #F6886) and was incubated for 30 min at 37° C. with 5% CO2. Then, 20 μL/well of detection solution was added, prepared as instructed in the HitHunter® cAMP Assay for Small Molecules Kit, the plate was wrapped in foil, and was incubated for 1 hr at room temperature. Then, 20 μL/well of Solution A from the HitHunter® cAMP Assay for Small Molecules kit was added and was incubated in foil at room temperature overnight. Plates were read after ˜8 hrs of incubation on a standard luminescence plate reader. In this assay, agonist activity was inhibitory due to the use of forskolin, therefore low luminescence signal was directly related to high cAMP activity. Using Four Parameter Logistic Regression for curve fitting, the raw data file was processed into Z-shaped dose-response curves from which values, such as the EC50, Hill Slope, and Emax, were quantified.

### β-Arrestin Signaling Assay

In a 384-well plate (Corning #3897), PathHunter® CHO-K1 AGTRL1 β-arrestin cells (Eurofins DiscoverX #93-0250C2) were seeded at 5×103 cells/well in 20 μL AssayComplete Cell Plating 2 Reagent (Eurofins DiscoverX #93-0563R2A) and were incubated overnight at 37° C. with 5% CO2. On the following day, the plate was treated with 5 μL/well of compounds serially diluted in 1×PBS+0.1% BSA buffer and was incubated for 1.5 hr at 37° C. with 5% CO2. Then, 12.5 μL/well of detection solution was added, prepared as instructed in the PathHunter® Detection Kit (Eurofins DiscoverX #93-0001), the plate was wrapped in foil, and was incubated at room temperature. Plates were read after 1-2 hr of incubation on a standard luminescence plate reader. In this assay, low levels of β-arrestin in cells were directly related to low luminescence signal. Using Four Parameter Logistic Regression for curve fitting, the raw data file was processed into S-shaped dose-response curves from which values, such as the EC50, Hill Slope, and Emax, were quantified.

The EC50 values determined from the above-explained three assays are summarized in Table 4. Proteolysis during production was detected using mass spectroscopy, which provides insights into the proteolytic stability of the apelin receptor agonists.

TABLE 4
Summary of EC50 values

| | Apelin | | | | | ERK | Proteolysis |
|---|---|---|---|---|---|---|---|
| SEQ ID | Receptor | cAMP EC50 (nM) | | beta-Arrestin EC50 (nM) | | EC50 | During |
| NO. | Agonist | Exp. 1 | Exp. 2 | Exp. 1 | Exp. 2 | (nM) | Production? |
| | Apelin-13 | | 0.7 | | 3.8 | | |
| | Elabela-22 | | 34 | | 10.6 | | |
| 17 | APL-A | 6.85 | 2.2 | 1009 | Weak | 1.97 | Yes |
| 18 | APL-B | 3.658 | 5.4 | 337.1 | 109.6 | 2.63 | No |
| 19 | APL-C | 50.37 | 24.621 | Weak | Weak | 0.68 | |
| 20 | APL-D | 3.45 | 11.088 | 180.7 | Weak | 0.1 | |
| 21 | APL-E | 54.65 | 37.5 | Weak | Weak | 14.36 | Yes |
| 22 | APL-F | 18.31 | 13.6 | 1426 | Weak | 1.954 | No |
| 23 | APL-G | 2.806 | 1.2 | 23.59 | 44.5 | 0.64 | No |
| 24 | ELA-A | 0.85 | 1.4 | 90.13 | 42.6 | 0.19 | No |
| 25 | ELA-B | 0.349 | 1.5 | 1120 | 59.9 | 0.92 | No |
| 26 | ELA-C | 0.577 | 1 | 535.1 | 75.1 | 1.13 | No |
| 27 | ELA-D | 10.7 | 4.4 | Weak | 921.9 | | No |
| 28 | ELA-E | 4.42 | 2.4 | Weak | 352 | | No |
| 29 | ELA-F | 1.01 | 1 | 9.59 | 43.5 | | No |
| 36 | ELA-G | 9.91 | 14.3 | 89.073 | 521.2 | | No |
| 37 | ELA-H | 10.029 | 5.8 | 21.865 | 97.5 | | No |
| 38 | ELA-I | 22.917 | 3.195 | 17.258 | 150.26 | | Yes |
| 39 | ELA-J | 18.564 | 133.46 | 73.74 | Weak | | Yes |
| 40 | ELA-K | 4.759 | 5.7 | 3.448 | 12.2 | | No |
| 41 | ELA-L | 0.632 | 2.8 | 7.538 | 6.9 | | No |
| 50 | APL-H | 6.540 | 6.5 | 172.4 | 172.4 | | No |
| 51 | APL-I | 18.039 | 18 | Weak | Weak | | No |
| 52 | APL-J | 3.796 | 3.8 | 370 | 372.6 | | No |
| 53 | APL-K | 14.239 | 14.2 | Weak | Weak | | No |
| 54 | APL-L | 73.432 | 73.4 | Weak | Weak | | No |
| 55 | APL-M | 8.298 | 8.3 | Weak | Weak | | No |

### Example 2—Measurement of Desensitization by Apelin Receptor Agonists

Persistent apelin receptor signaling can lead to transcriptional downregulation of the receptor and its downstream effectors. This causes the removal of the receptor from the cell membrane or the decoupling of receptor activation to downstream effector functions, respectively, once again decreasing the effectiveness of both the natural ligands and drugs. The present invention provides, among other things, superior apelin receptor agonists that have reduced desensitization, which provides improved efficacy and potency.

The extent of desensitization caused by the apelin receptor's natural ligands and the apelin receptor agonists of the present invention is assessed through following assays:
1. Measure the efficacy and potency of β-arrestin signaling.
2. Use of microscopy to measure track the localization of the apelin receptor, with the aim of quantifying the degree of receptor internalization that occurs after treatment.
3. Measure the level of transcriptional downregulation of the apelin receptor and selected downstream signaling molecules using RT-PCR.
4. Perform repeated treatments and measurements of apelin receptor agonists in in vitro and in vivo assays to see if the same magnitude of response is obtained on later measurements.

### Example 3—Internalization of Apelin Receptor Agonists

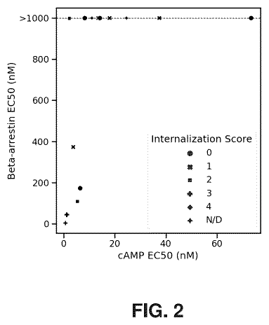

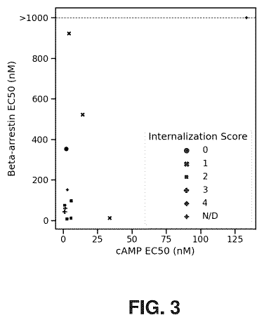

In this example, the internalization of exemplary apelin receptor agonist was assessed. CHO-M1 cells expressing an APJ-eGFP fusion protein were stained and plated in a 96-well plate. Apelin receptor agonists were serially diluted and added to the wells at concentrations ranging from 0.914 nM to 2000 nM. The cells were later imaged using an Operetta CLS™ high-content analysis system. APJ-eGFP signal was measured in the membrane and cytoplasmic region, and the ratio of membrane signal to total signal was calculated (E ratio). An internalization score was assigned based on the lowest concentration of the compound at which maximum internalization was observed. Scores were assigned according to Table 5, and the internalization scores are summarized in Table 6. The internalization score for each apelin receptor agonist was plotted against the EC50 values for cAMP signaling and beta-arrestin signaling as shown in FIG. 2 and FIG. 3.

TABLE 5
Internalization Score Values

| Internalization Score | Concentration Range | |
|---|---|---|
| 0 | No Internalization | |
| 1 | >1000 | nM |
| 2 | 100-1000 | nM |
| 3 | 10-100 | nM |
| 4 | <10 | nM |

| | |
|---|---|
| N/D | Not Determined |

TABLE 6
Internalization Scores for Exemplary Apelin Receptor Agonists

| SEQ ID NO. | Apelin Receptor Agonist | Internalization Score |
|---|---|---|
| 1 | Apelin-13 | 3 |
| 17 | APL-A | 2 |
| 18 | APL-B | 2 |
| 19 | APL-C | N/D |
| 20 | APL-D | N/D |
| 21 | APL-E | 1 |
| 22 | APL-F | 1 |
| 23 | APL-G | 3 |
| 50 | APL-H | 0 |
| 51 | APL-I | 1 |
| 52 | APL-J | 1 |
| 53 | APL-K | 0 |
| 54 | APL-L | 0 |
| 55 | APL-M | 0 |
| 8 | Elabela-22 | 1 |
| 24 | ELA-A | 3 |
| 25 | ELA-B | 3 |
| 26 | ELA-C | 2 |
| 27 | ELA-D | 1 |
| 28 | ELA-E | 0 |
| 29 | ELA-F | 3 |
| 36 | ELA-G | 1 |
| 37 | ELA-H | 2 |
| 38 | ELA-I | N/D |
| 39 | ELA-J | N/D |
| 40 | ELA-K | 2 |
| 41 | ELA-L | 2 |

1. An apelin receptor agonist comprising an apelin analogue, wherein the apelin analogue comprises one or more amino acid substitutions as compared to SEQ ID NO: 1 (QRPRLSHKGPMPF),
wherein the one or more amino acid substitution occurs at position 1, 6, 8, 9, 11, 12, and/or 13.

2. The apelin receptor agonist of claim 1, wherein the one or more amino acid substitutions comprises P12V, K8W, Q1E, M11H, M11A, F13Y, P12A, P12H, F13W, S6Q, and/or G9N.

3. An apelin receptor agonist comprising an apelin analogue, wherein the apelin analogue comprises the amino sequence:
X1-R-P-R-L-S-H-X2-G-P-X3-X4-X5
wherein
X1 is Q or E;
X2 is K or W;
X3 is M, H, or A;
X4 is P, V, A, or H; and/or
X5 is F, Y, or W.

4. The apelin receptor agonist of claim 1, wherein the apelin analogue comprises the amino sequence:
Q-R-P-R-L-Q-H-K-N-P-M-A-X1
wherein
X1 is F or W.

5. The apelin receptor agonist of claim 1, wherein the apelin analogue comprises the amino sequence:
Q-R-P-R-L-S-H-K-G-P-X1-X2-X3
wherein
X1 is M, H, or A;
X2 is P, V, A, or H; and/or
X3 is F, Y, or W.

6. The apelin receptor agonist of claim 1, wherein the apelin analogue comprises the amino sequence:
X1-R-P-R-L-X2-H-X3-X4-P-X5-X6-X7
wherein
X1 is Q or E;
X2 is S or Q;
X3 is K or W;
X4 is G or N;
X5 is M, H, or A;
X6 is P, V, A or H; and/or
X7 is F, Y or W.

7. The apelin receptor agonist of claim 1, wherein the apelin analogue comprises the amino acid sequence of any one of SEQ ID NOs: 1-7 or 50-55.

8-19. (canceled)

20. An apelin receptor agonist comprising an elabela analogue, wherein the elabela analogue comprises one or more amino acid substitutions as compared to SEQ ID NO: 8
(KLRKHNCLQRRCMPLHSRVPFP),
wherein the one or more amino acid substitution occurs at position 3, 7, 8, 9, 10, 12, 14, 15, 17 and/or 18.

21. The apelin receptor agonist of claim 20, wherein the one or more amino acid substitutions comprise R3N, P14I, S17Q, S17R, Q9K, R18Y, L15Y, C7S, C12Y, R10E, Q9I, L8A, and/or Q9T.

22. The apelin receptor agonist of claim 20, wherein the elabela analogue comprises the amino sequence:
K-L-Z1-K-H-N-Z2-Z3-Z4-Z5-R-
Z6-M-Z7-Z8-H-Z9-Z10-V-P-F-P
wherein
Z1 is R or N;
Z2 is C or S;
Z3 is A or L;
Z4 is Q, I, K, or T;
Z5 is R or E;
Z6 is C or Y;
Z7 is P or I;
Z8 is L or Y;
Z9 is S, Q, or R; and/or
Z10 is R or Y.

23. The apelin receptor agonist of claim 20, wherein the elabela analogue comprises the amino acid sequence of any one of SEQ ID NOs: 8-13 or 30-35.

24-34. (canceled)

35. The apelin receptor agonist of claim 1, wherein the apelin analogue is fused to a carrier moiety.

36-45. (canceled)

46. The apelin receptor agonist of claim 35, wherein the carrier moiety comprises an Fc domain, a human serum albumin (HSA), an anti-HSA antibody or a fragment thereof, lipids, or PEGs.

47-85. (canceled)

86. A method of treating a disease comprising administering the apelin receptor agonist of claim 1 to a subject in need thereof.

87. (canceled)

88. A nucleic acid encoding the apelin receptor agonist of the preceding claim 1.

89. A method of producing the apelin receptor agonist of the preceding claim 1.

90. The apelin receptor agonist of claim 20, wherein the elabela analogue is fused to a carrier moiety, and wherein the carrier moiety comprises an Fc domain, a human serum albumin (HSA), an anti-HSA antibody or a fragment thereof, lipids, or PEGs.

91. A method of treating a disease comprising administering the apelin receptor agonist of claim 20 to a subject in need thereof.

92. A nucleic acid encoding the apelin receptor agonist of claim 20.

93. A method of producing the apelin receptor agonist of claim 20.


SEQUENCE LISTING (67 sequences)

SEQ ID NO: 1 - protein, 13 aa, synthetic construct
QRPRLSHKGPMPF

SEQ ID NO: 2 - protein, 13 aa, synthetic construct
QRPRLSHKGPMVF

SEQ ID NO: 3 - protein, 13 aa, synthetic construct
QRPRLSHWGPMPF

SEQ ID NO: 4 - protein, 13 aa, synthetic construct
ERPRLSHKGPMPF

SEQ ID NO: 5 - protein, 13 aa, synthetic construct
QRPRLSHKGPHPF

SEQ ID NO: 6 - protein, 13 aa, synthetic construct
QRPRLSHKGPAPY

SEQ ID NO: 7 - protein, 13 aa, synthetic construct
QRPRLSHKGPMAF

SEQ ID NO: 8 - protein, 22 aa, synthetic construct
KLRKHNCLQRRCMPLHSRVPFP

SEQ ID NO: 9 - protein, 22 aa, synthetic construct
KLNKHNCLQRRCMPLHSRVPFP

SEQ ID NO: 10 - protein, 22 aa, synthetic construct
KLRKHNCLQRRCMILHSRVPFP

SEQ ID NO: 11 - protein, 22 aa, synthetic construct
KLRKHNCLQRRCMPLHQRVPFP

SEQ ID NO: 12 - protein, 22 aa, synthetic construct
KLRKHNCLQRRCMPLHRRVPFP

SEQ ID NO: 13 - protein, 22 aa, synthetic construct
KLRKHNCLKRRCMPLHSRVPFP

SEQ ID NO: 14 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 15 - protein, 244 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 16 - protein, 15 aa, synthetic construct
GGGGSGGGGSGGGGS

SEQ ID NO: 17 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPMPF

SEQ ID NO: 18 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPMVF

SEQ ID NO: 19 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHWGPMPF

SEQ ID NO: 20 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSERPRLSHKGPMPF

SEQ ID NO: 21 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPHPF

SEQ ID NO: 22 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPAPY

SEQ ID NO: 23 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPMAF

SEQ ID NO: 24 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQRRCMPLHSRVPFP

SEQ ID NO: 25 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLNKHNCLQRRCMPLHSRVPFP

SEQ ID NO: 26 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQRRCMILHSRVPFP

SEQ ID NO: 27 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQRRCMPLHQRVPFP

SEQ ID NO: 28 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQRRCMPLHRRVPFP

SEQ ID NO: 29 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLKRRCMPLHSRVPFP

SEQ ID NO: 30 - protein, 22 aa, synthetic construct
KLRKHNCLQRRCMPLHSYVPFP

SEQ ID NO: 31 - protein, 22 aa, synthetic construct
KLRKHNCLQRRCMPYHSRVPFP

SEQ ID NO: 32 - protein, 22 aa, synthetic construct
KLRKHNSLQRRYMPLHSRVPFP

SEQ ID NO: 33 - protein, 22 aa, synthetic construct
KLRKHNCLQERCMPLHSRVPFP

SEQ ID NO: 34 - protein, 22 aa, synthetic construct
KLRKHNCLIRRCMPLHSRVPFP

SEQ ID NO: 35 - protein, 22 aa, synthetic construct
KLRKHNCATRRCMPLHSRVPFP

SEQ ID NO: 36 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQRRCMPLHSYVPFP

SEQ ID NO: 37 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQRRCMPYHSRVPFP

SEQ ID NO: 38 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNSLQRRYMPLHSRVPFP

SEQ ID NO: 39 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLQERCMPLHSRVPFP

SEQ ID NO: 40 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCLIRRCMPLHSRVPFP

SEQ ID NO: 41 - protein, 281 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSKLRKHNCATRRCMPLHSRVPFP

SEQ ID NO: 42 - protein, 77 aa, synthetic construct
MNLRLCVQALLLLWLSLTAVCGGSLMPLPDGNGLEDGNVRHLVQPRGSRNGPGPWQGGRRKFRRQRPRLSHKGPMPF

SEQ ID NO: 43 - protein, 36 aa, synthetic construct
LVQPRGSRNGPGPWQGGRRKFRRQRPRLSHKGPMPF

SEQ ID NO: 44 - protein, 17 aa, synthetic construct
KFRRQRPRLSHKGPMPF

SEQ ID NO: 45 - protein, 13 aa, synthetic construct
ERPRLSHKGPMPF

SEQ ID NO: 46 - protein, 54 aa, synthetic construct
MRFQQFLFAFFIFIMSLLLISGQRPVNLTMRRKLRKHNCLQRRCMPLHSRVPFP

SEQ ID NO: 47 - protein, 32 aa, synthetic construct
QRPVNLTMRRKLRKHNCLQRRCMPLHSRVPFP

SEQ ID NO: 48 - protein, 21 aa, synthetic construct
LRKHNCLQRRCMPLHSRVPFP

SEQ ID NO: 49 - protein, 11 aa, synthetic construct
CMPLHSRVPFP

SEQ ID NO: 50 - protein, 13 aa, synthetic construct
QRPRLSHKGPMHW

SEQ ID NO: 51 - protein, 13 aa, synthetic construct
QRPRLSHKGPAAW

SEQ ID NO: 52 - protein, 13 aa, synthetic construct
QRPRLSHKGPAAY

SEQ ID NO: 53 - protein, 13 aa, synthetic construct
QRPRLQHKNPMAF

SEQ ID NO: 54 - protein, 13 aa, synthetic construct
QRPRLQHKNPMAW

SEQ ID NO: 55 - protein, 13 aa, synthetic construct
QRPRLSHWGPMAW

SEQ ID NO: 56 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPMHW

SEQ ID NO: 57 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPAAW

SEQ ID NO: 58 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHKGPAAY

SEQ ID NO: 59 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLQHKNPMAF

SEQ ID NO: 60 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLQHKNPMAW

SEQ ID NO: 61 - protein, 272 aa, synthetic construct
MRAWIFFLLCLAGRALADKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGKGGGGSGGGGSGGGGSQRPRLSHWGPMAW

SEQ ID NO: 62 - protein, 17 aa, synthetic construct
MRAWIFFLLCLAGRALA

SEQ ID NO: 63 - protein, 13 aa, synthetic construct
  VARIANT 1: note = X = Q or E
  VARIANT 8: note = X = K or W
  VARIANT 11: note = X = M or H or A
  VARIANT 12: note = X = P or V or A or H
  VARIANT 13: note = X = F or Y or W
XRPRLSHXGPXXX

SEQ ID NO: 64 - protein, 13 aa, synthetic construct
  VARIANT 13: note = X = F or W
QRPRLQHKNPMAX

SEQ ID NO: 65 - protein, 13 aa, synthetic construct
  VARIANT 11: note = X = M or H or A
  VARIANT 12: note = X = P or V or A or H
  VARIANT 13: note = X = F or Y or W
QRPRLSHKGPXXX

SEQ ID NO: 66 - protein, 13 aa, synthetic construct
  VARIANT 1: note = X = Q or E
  VARIANT 6: note = X = S or Q
  VARIANT 8: note = X = K or W
  VARIANT 9: note = X = G or N
  VARIANT 11: note = X = M or H or A
  VARIANT 12: note = X = P or V or A or H
  VARIANT 13: note = X = F or Y or W
XRPRLXHXXPXXX

SEQ ID NO: 67 - protein, 22 aa, synthetic construct
  VARIANT 3: note = X = R or N
  VARIANT 7: note = X = C or S
  VARIANT 8: note = X = A or L
  VARIANT 9: note = X = Q or I or K or T
  VARIANT 10: note = X = R or E
  VARIANT 12: note = X = C or Y
  VARIANT 14: note = X = P or I
  VARIANT 15: note = X = L or Y
  VARIANT 17: note = X = S or Q or R
  VARIANT 18: note = X = R or Y
KLXKHNXXXXRXMXXHXXVPFP